(19) United States
(12) Patent Application Publication
(10) Pub. No.: US 2021/0375460 A1
(43) Pub. Date: Dec. 2, 2021

(54) ACCURATE PREDICTION AND TREATMENT OF MYOPIC PROGRESSION BY ARTIFICIAL INTELLIGENCE

(71) Applicants: Zhihuan LI, Shenyang (CN); AI TECHNOLOGIES INC., Grand Cayman (KY)

(72) Inventors: Zhihuan LI, Shenyang (CN); Rui HOU, Shenyang (CN); Lianghong ZHENG, Shenyang (CN)

(21) Appl. No.: 17/240,088

(22) Filed: Apr. 26, 2021

Related U.S. Application Data
(63) Continuation of application No. PCT/CN2019/113325, filed on Oct. 25, 2019.
(60) Provisional application No. 62/751,171, filed on Oct. 26, 2018.

(51) Int. Cl. G16H 50/20; G16H 20/10; G16H 20/30; G06N 20/10

(52) U.S. Cl. CPC G16H 50/20; G06N 20/10; G16H 20/30; G16H 20/10

(57) ABSTRACT

Disclosed herein are systems, methods, devices, and media for carrying out diagnosis of myopia onset and progression. Machine learning algorithms enable the automated analysis of relevant features to generate predictions. Also disclosed are treatment methods incorporating the machine learning algorithms to identify suitable treatments and predict treatment efficacy.

### CROSS-REFERENCE TO RELATED APPLICATIONS

This application is a continuation of International Application No. PCT/CN2019/113325, filed Oct. 25, 2019, which claims the benefit of U.S. Provisional Application No. 62/751,171, filed Oct. 26, 2018, the disclosure of each of which is incorporated herein by reference in its entirety.

### BACKGROUND OF THE DISCLOSURE

Myopia is the leading cause of visual impairment across the globe. In 2016, the prevalence of myopia reached nearly 1.6 billion cases worldwide, a trend expected to surpass 5.6 billion cases within the next few decades. China's population alone hosts nearly 400 million myopic cases. In recent years, it has also become the most common form of visual impairment in Asian school children.

### SUMMARY OF THE DISCLOSURE

Disclosed herein are systems, methods, media, and devices providing Artificial Intelligence (AI) for making predictions or diagnoses of myopia onset and/or progression. The AI disclosed herein has the potential to revolutionize disease diagnosis and prediction, leading to a better standard of care.

In some embodiments, the systems and methods disclosed herein account for variations in myopic distribution. For example, myopic distribution has been shown to vary amongst different racial and environmental backgrounds. Typical onset occurs between 5-15 years of age, but recent Chinese reports have noted development within the first six months of birth. Studies have shown that children from China, Singapore, and Taiwan have drastically higher prevalence rates of myopia than their European counterparts, despite studies reporting equal distributions amongst Chinese and European adult populations. For example, it has been found that 3.4% of 10-11-year-old UK school children presented with myopia compared to 30.1% of 10-year-old urban Chinese school children. Despite this 10-fold difference, the exponential prevalence in the years following raises a public health concern. By age 15, 78.4% of urban Chinese students have acquired myopia, by age 18, this frequency reaches to 80%. Other Asian countries, such as South Korean and Taiwan have also noted similar trends with the prevalence of myopia occurring in 96.5% and 90% of 19-year-old Seoul men and Taiwanese University Students respectively. Besides the growing prevalence of myopia over time, studies show there is an increase in the amount of myopia from childhood age to young adults, suggesting progression of myopia.

Accordingly, disclosed herein are systems and methods for predicting myopia onset and/or progression for an individual belonging to a target population. In some embodiments, the target population corresponds to an ethnic group, a nationality, a geographic location or area, or other factor common to a population. In some embodiments, the systems and methods incorporate population information into the predictive algorithm used to predict myopia onset and/or progression. For example, a predictive algorithm can comprise one or more features corresponding to ethnicity and/or nationality of an individual in predicting myopia onset and/or progression for the individual. Alternatively, or in combination, a predictive algorithm can be trained on a specific population such that an individual can be screened and then evaluated by the appropriate algorithm trained on the appropriate population data.

Myopia's ongoing progression can produce a number of complications from cataracts, glaucoma, chorioretinal degeneration, to more severe comorbidities such as macular degeneration or retinal detachment that can lead to permanent visual impairment. The recent surge in myopia progression in Asian school children has left some to claim the emergence of a “myopic epidemic”. The average economic cost to prevent myopic progression in Singaporean adults equates to $US709 per year. In the US alone, $250 million in annual funds are spent on treatment, while global refractive care services approach $20 billion. The economic burden of correcting myopic visual impairment places a large population at risk for not seeking treatment, and it is estimated that $121.4 billion in global funds are lost due to ongoing uncorrected visual impairment. In fact, studies have shown that visual impairment is correlated with reduced economic productivity, reduced quality of life, and even increased mortality. Therefore, providing a predictive analysis of myopic progression would not only mitigate visual impairment but also relieve both personal and economic restraints placed on the patient, since preventive measures could be taken to avoid visual impairment due to progressive myopia.

The increasing prevalence over the past few decades, the overwhelming amount of untreated refractive errors and the potential risk of permanent visual impairment show the vital need to prevent and treat myopia progression. As such, the World Health Organization has established the VISION 2020 global initiation to eliminate myopia and other leading causes of avoidable blindness. Current methods to treat myopia involve the use of spectacles, corneal reshaping contact lenses, pharmacological agents and corneal refractive surgery; however, not all methods are able to prevent the rate of progression. Reports have shown that the introductive measures at an early age can drastically reduce the progression of myopia, and possibly reduce future economic burden. In separate studies conducted by Cho and Walline, corneal reshaping contact lenses have shown to slow the degree of progression by nearly 50%. Similar results have been shown with the use of Atropine in school children.

Accordingly, disclosed herein are systems and methods for detecting myopia onset and/or progression using artificial intelligence that allow for treating or preventing myopia onset and/or progression. In some embodiments, the systems and methods disclosed herein comprise steps for treating or preventing myopia onset and/or progression. In some embodiments, the treatment and/or prevention is personalized based on the associated prediction or evaluation of myopia onset and/or progression. For example, given a prediction of myopia onset when myopia has not yet occurred or when myopia is in the early stages, a recommended prevention step may be lifestyle changes such as increasing outdoor activities or exposure to sunlight, or reducing near-work such as reading. In another example, given a prediction of myopia progression, a recommended step for preventing or reducing myopia progression can be the use of atropine eye drops.

In one aspect, disclosed herein is a computer-implemented method for predicting myopia progression in an individual, comprising: a) obtaining data of the individual; b) evaluating the data using a machine learning algorithm to generate a prediction of myopia progression; and c) providing the prediction to the individual or a third party. In some embodiments, the machine learning algorithm comprises a linear model providing a relationship between myopia progression and two or more features. In some embodiments, the machine learning algorithm is generated using a machine learning procedure comprising multivariate linear regression analysis. In some embodiments, the machine learning algorithm comprises a logistic regression model. In some embodiments, the machine learning algorithm comprises a support vector machine model. In some embodiments, the prediction comprises a likelihood of progression to a higher degree of myopia. In some embodiments, the higher degree of myopia is low myopia, moderate myopia, or high myopia. In some embodiments, the low myopia corresponds to a spherical equivalent of −3.00 diopters or less. In some embodiments, the moderate myopia corresponds to a spherical equivalent between −3.00 diopters to −6.00 diopters. In some embodiments, the high myopia corresponds to a spherical equivalent of −6.00 diopters or more. In some embodiments, the individual has myopia. In some embodiments, the individual does not have myopia. In some embodiments, the prediction comprises an age of onset of myopia in an individual who does not have myopia. In some embodiments, the prediction comprises a rate of progression of myopia. In some embodiments, the rate of progression comprises a predicted change in degree of myopia within a time period. In some embodiments, the time period is no more than about 6 months, about 1 year, about 2 years, about 3 years, about 4 years, about 5 years, about 6 years, about 7 years, about 8 years, about 9 years, or no more than about 10 years. In some embodiments, the predicted change in degree of myopia is about 1 diopters or less, from about 1 to about 2 diopters, from about 2 to about 3 diopters, from about 3 to about 4 diopters, from about 4 to about 5 diopters, from about 5 to about 6 diopters, from about 6 to about 7 diopters, from about 7 to about 8 diopters, from about 8 to about 9 diopters, from about 9 to about 10 diopters, from about 10 to about 12 diopters, from about 12 to about 15 diopters, from about 15 to about 20 diopters, from about 20 to about 30 diopters, from about 30 to about 40 diopters, or from about 40 to about 50 diopters. In some embodiments, the prediction comprises a predicted change in myopia for one or both eyes of the individual. In some embodiments, the data comprises an age and refraction under cycloplegia of one or both eyes of the individual measured at one or more time points. In some embodiments, the one or more time points comprises at least 2, at least 3, at least 4, at least 5, at least 6, at least 7, at least 8, at least 9, or at least 10 time points. In some embodiments, the method further comprises processing the data to calculate a spherical equivalent based on the refraction. In some embodiments, the prediction has an accuracy of at least 80% when tested against an independent data set of at least 200 samples. In some embodiments, the method further comprises providing a recommendation for treatment to the individual or the third party. In some embodiments, the method further comprises prescribing a treatment to the individual. In some embodiments, the method further comprises providing a treatment to the individual. In some embodiments, the treatment comprises atropine eye drops. In some embodiments, the treatment comprises orthokeratology. In some embodiments, the method further comprises performing further testing on the individual. In some embodiments, the further testing comprises an optometry exam, genetic testing, imaging one or both eyes of the individual, screening for ocular disorders or conditions, or any combination thereof. In some embodiments, the individual no more than 5 years old, 6 years old, 7 years old, 8 years old, 9 years old, 10 years old, 11 years old, 12 years old, 13 years old, 14 years old, 15 years old, 16 years old, 17 years old, 18 years old, 19 years old, 20 years old, 21 years old, 22 years old, 23 years old, 24 years old, 25 years old, 26 years old, 27 years old, 28 years old, 29 years old, or no more than 30 years old. In some embodiments, the individual is male or female. In some embodiments, the data comprises average exposure to sunlight or natural lighting, intensity of sunlight exposure, average UV index, latitude, or any combination thereof.

In another aspect, disclosed herein is a computer-implemented system comprising: a) an electronic device comprising: a processor, a memory, and an operating system configured to perform executable instructions; and b) a computer program stored in the memory of the electronic device, the computer program including instructions executable by the user electronic device to create an application comprising: i) a software module obtaining data of the individual; ii) a software module evaluating the data using a machine learning algorithm to generate a prediction of myopia progression; and iii) a software module providing the prediction to the individual or a third party. In some embodiments, the machine learning algorithm comprises a linear model providing a relationship between myopia progression and two or more features. In some embodiments, the machine learning algorithm is generated using a machine learning procedure comprising multivariate linear regression analysis. In some embodiments, the machine learning algorithm comprises a logistic regression model. In some embodiments, the machine learning algorithm comprises a support vector machine model. In some embodiments, the prediction comprises a likelihood of progression to a higher degree of myopia. In some embodiments, the higher degree of myopia is low myopia, moderate myopia, or high myopia. In some embodiments, the low myopia corresponds to a spherical equivalent of −3.00 diopters or less. In some embodiments, the moderate myopia corresponds to a spherical equivalent between −3.00 diopters to −6.00 diopters. In some embodiments, the high myopia corresponds to a spherical equivalent of −6.00 diopters or more. In some embodiments, the individual has myopia. In some embodiments, the individual does not have myopia. In some embodiments, the prediction comprises an age of onset of myopia in an individual who does not have myopia. In some embodiments, the prediction comprises a rate of progression of myopia. In some embodiments, the rate of progression comprises a predicted change in degree of myopia within a time period. In some embodiments, the time period is no more than about 6 months, about 1 year, about 2 years, about 3 years, about 4 years, about 5 years, about 6 years, about 7 years, about 8 years, about 9 years, or no more than about 10 years. In some embodiments, the predicted change in degree of myopia is about 1 diopters or less, from about 1 to about 2 diopters, from about 2 to about 3 diopters, from about 3 to about 4 diopters, from about 4 to about 5 diopters, from about 5 to about 6 diopters, from about 6 to about 7 diopters, from about 7 to about 8 diopters, from about 8 to about 9 diopters, from about 9 to about 10 diopters, from about 10 to about 12 diopters, from about 12 to about 15 diopters, from about 15 to about 20 diopters, from about 20 to about 30 diopters, from about 30 to about 40 diopters, or from about 40 to about 50 diopters. In some embodiments, the prediction comprises a predicted change in myopia for one or both eyes of the individual. In some embodiments, the data comprises an age and refraction under cycloplegia of one or both eyes of the individual measured at one or more time points. In some embodiments, the one or more time points comprises at least 2, at least 3, at least 4, at least 5, at least 6, at least 7, at least 8, at least 9, or at least 10 time points. In some embodiments, the application further comprises a software module processing the data to calculate a spherical equivalent based on the refraction. In some embodiments, the prediction has an accuracy of at least 80% when tested against an independent data set of at least 200 samples. In some embodiments, the application further comprises a software module providing a recommendation for treatment to the individual or the third party. In some embodiments, the application further comprises a software module prescribing a treatment to the individual. In some embodiments, the treatment comprises atropine eye drops. In some embodiments, the treatment comprises orthokeratology. In some embodiments, the application further comprises a software module providing instructions to perform further testing on the individual. In some embodiments, the further testing comprises an optometry exam, genetic testing, imaging one or both eyes of the individual, screening for ocular disorders or conditions, or any combination thereof. In some embodiments, the individual is no more than 5 years old, 6 years old, 7 years old, 8 years old, 9 years old, 10 years old, 11 years old, 12 years old, 13 years old, 14 years old, 15 years old, 16 years old, 17 years old, 18 years old, 19 years old, 20 years old, 21 years old, 22 years old, 23 years old, 24 years old, 25 years old, 26 years old, 27 years old, 28 years old, 29 years old, or no more than 30 years old. In some embodiments, the individual is male or female. In some embodiments, the data comprises average exposure to sunlight or natural lighting, intensity of sunlight exposure, average UV index, latitude, or any combination thereof.

In another aspect, disclosed herein is non-transitory computer readable medium including instructions executable by a processor to create an application comprising: a) a software module obtaining data of the individual; b) a software module evaluating the data using a machine learning algorithm to generate a prediction of myopia progression; and c) a software module providing the prediction to the individual or a third party. In some embodiments, the machine learning algorithm comprises a linear model providing a relationship between myopia progression and two or more features. In some embodiments, the machine learning algorithm is generated using a machine learning procedure comprising multivariate linear regression analysis. In some embodiments, the machine learning algorithm comprises a logistic regression model. In some embodiments, the machine learning algorithm comprises a support vector machine model. In some embodiments, the prediction comprises a likelihood of progression to a higher degree of myopia. In some embodiments, the higher degree of myopia is low myopia, moderate myopia, or high myopia. In some embodiments, the low myopia corresponds to a spherical equivalent of −3.00 diopters or less. In some embodiments, the moderate myopia corresponds to a spherical equivalent between −3.00 diopters to −6.00 diopters. In some embodiments, the high myopia corresponds to a spherical equivalent of −6.00 diopters or more. In some embodiments, the individual has myopia. In some embodiments, the individual does not have myopia. In some embodiments, the prediction comprises an age of onset of myopia in an individual who does not have myopia. In some embodiments, the prediction comprises a rate of progression of myopia. In some embodiments, the rate of progression comprises a predicted change in degree of myopia within a time period. In some embodiments, the time period is no more than about 6 months, about 1 year, about 2 years, about 3 years, about 4 years, about 5 years, about 6 years, about 7 years, about 8 years, about 9 years, or no more than about 10 years. In some embodiments, the predicted change in degree of myopia is about 1 diopters or less, from about 1 to about 2 diopters, from about 2 to about 3 diopters, from about 3 to about 4 diopters, from about 4 to about 5 diopters, from about 5 to about 6 diopters, from about 6 to about 7 diopters, from about 7 to about 8 diopters, from about 8 to about 9 diopters, from about 9 to about 10 diopters, from about 10 to about 12 diopters, from about 12 to about 15 diopters, from about 15 to about 20 diopters, from about 20 to about 30 diopters, from about 30 to about 40 diopters, or from about 40 to about 50 diopters. In some embodiments, the prediction comprises a predicted change in myopia for one or both eyes of the individual. In some embodiments, the data comprises an age and refraction under cycloplegia of one or both eyes of the individual measured at one or more time points. In some embodiments, the one or more time points comprises at least 2, at least 3, at least 4, at least 5, at least 6, at least 7, at least 8, at least 9, or at least 10 time points. In some embodiments, the application further comprises a software module processing the data to calculate a spherical equivalent based on the refraction. In some embodiments, the prediction has an accuracy of at least 80% when tested against an independent data set of at least 200 samples. In some embodiments, the application further comprises a software module providing a recommendation for treatment to the individual or the third party. In some embodiments, the application further comprises a software module prescribing a treatment to the individual. In some embodiments, the treatment comprises atropine eye drops. In some embodiments, the treatment comprises orthokeratology. In some embodiments, the application further comprises a software module providing instructions to perform further testing on the individual. In some embodiments, the further testing comprises an optometry exam, genetic testing, imaging one or both eyes of the individual, screening for ocular disorders or conditions, or any combination thereof. In some embodiments, the individual is no more than 5 years old, 6 years old, 7 years old, 8 years old, 9 years old, 10 years old, 11 years old, 12 years old, 13 years old, 14 years old, 15 years old, 16 years old, 17 years old, 18 years old, 19 years old, 20 years old, 21 years old, 22 years old, 23 years old, 24 years old, 25 years old, 26 years old, 27 years old, 28 years old, 29 years old, or no more than 30 years old. In some embodiments, the individual is male or female. In some embodiments, the data comprises average exposure to sunlight or natural lighting, intensity of sunlight exposure, average UV index, latitude, or any combination thereof.

In another aspect, disclosed herein is a method for performing a personalized evaluation of an individual for myopia onset or myopia progression, comprising: a) obtaining input data of the individual; b) evaluating the input data using a machine learning model to generate a prediction of myopia onset or myopia progression, wherein the machine learning model has a sensitivity of at least about 90% and a specificity of at least about 90% when evaluated against an independent data set of at least 200 samples; and c) providing the prediction to the individual or a third party. In some embodiments, the machine learning model is a linear model providing a relationship between myopia progression and two or more features corresponding to the input data. In some embodiments, the machine learning model is generated using a machine learning procedure comprising multivariate linear regression analysis. In some embodiments, the machine learning model comprises a logistic regression model. In some embodiments, the machine learning model comprises a support vector machine model. In some embodiments, the prediction comprises a likelihood of progression to a higher degree of myopia. In some embodiments, the higher degree of myopia is low myopia, moderate myopia, or high myopia. In some embodiments, the low myopia corresponds to a spherical equivalent of −3.00 diopters or less. In some embodiments, the moderate myopia corresponds to a spherical equivalent between −3.00 diopters to −6.00 diopters. In some embodiments, the high myopia corresponds to a spherical equivalent of −6.00 diopters or more. In some embodiments, the individual has myopia. In some embodiments, the individual does not have myopia. In some embodiments, the prediction comprises an age of onset or time to onset of myopia in an individual who does not currently have myopia. In some embodiments, the prediction comprises a rate of progression of myopia. In some embodiments, the rate of progression comprises a predicted change in degree of myopia within a time period. In some embodiments, the time period is no more than about 6 months, about 1 year, about 2 years, about 3 years, about 4 years, about 5 years, about 6 years, about 7 years, about 8 years, about 9 years, or no more than about 10 years. In some embodiments, the predicted change in degree of myopia is about 1 diopters or less, from about 1 to about 2 diopters, from about 2 to about 3 diopters, from about 3 to about 4 diopters, from about 4 to about 5 diopters, from about 5 to about 6 diopters, from about 6 to about 7 diopters, from about 7 to about 8 diopters, from about 8 to about 9 diopters, from about 9 to about 10 diopters, from about 10 to about 12 diopters, from about 12 to about 15 diopters, from about 15 to about 20 diopters, from about 20 to about 30 diopters, from about 30 to about 40 diopters, or from about 40 to about 50 diopters. In some embodiments, the prediction comprises a predicted change in myopia for one or both eyes of the individual. In some embodiments, the data comprises an age and refraction, optionally under cycloplegia, of one or both eyes of the individual measured at one or more time points. In some embodiments, the one or more time points comprises at least 2, at least 3, at least 4, at least 5, at least 6, at least 7, at least 8, at least 9, or at least 10 time points. In some embodiments, the data is processed to calculate a spherical equivalent based on the refraction. In some embodiments, the prediction has an accuracy of at least 80% when tested against an independent data set of at least 200 samples. In some embodiments, a recommendation for treatment is provided to the individual or the third party. In some embodiments, a treatment is prescribed to the individual. In some embodiments, a treatment is provided to the individual. In some embodiments, the treatment comprises atropine eye drops. In some embodiments, the treatment comprises orthokeratology. In some embodiments, the treatment comprises atropine eye drops, multifocal contact lenses, orthokeratology, multifocal eyeglasses, or any combination thereof. In some embodiments, further testing is performed on the individual for one or more ophthalmic conditions or disorders associated with myopia. In some embodiments, the one or more ophthalmic conditions or disorders comprises cataract, glaucoma, myopic maculopathy, retinal detachment, macular edema, choroidal neovascularization, staphyloma, or any combination thereof. In some embodiments, the further testing comprises an optometry exam, genetic testing, imaging one or both eyes of the individual, screening for ocular disorders or conditions, or any combination thereof. In some embodiments, the individual is no more than 5 years old, 6 years old, 7 years old, 8 years old, 9 years old, 10 years old, 11 years old, 12 years old, 13 years old, 14 years old, 15 years old, 16 years old, 17 years old, 18 years old, 19 years old, 20 years old, 21 years old, 22 years old, 23 years old, 24 years old, 25 years old, 26 years old, 27 years old, 28 years old, 29 years old, or no more than 30 years old. In some embodiments, the individual is male or female. In some embodiments, the data comprises average exposure to sunlight or natural lighting, intensity of sunlight exposure, average UV index, latitude, or any combination thereof. In some embodiments, the input data is obtained from an eye examination. In some embodiments, the input data comprises age, gender, nationality, ethnicity, spherical equivalent (SE) measurement of one or both eyes, In some embodiments, the machine learning model is a classifier. In some embodiments, the machine learning model is a regression model.

In another aspect, disclosed herein is a computer-implemented system comprising: c) an electronic device comprising: a processor, a memory, and an operating system configured to perform executable instructions; and d) a computer program stored in the memory of the electronic device, the computer program including instructions executable by the user electronic device to create an application comprising: i) a software module obtaining input data of the individual; ii) a software module evaluating the input data using a machine learning model to generate a prediction of myopia onset or myopia progression, wherein the machine learning model has a sensitivity of at least about 90% and a specificity of at least about 90% when evaluated against an independent data set of at least 200 samples; and iii) a software module providing the prediction to the individual or a third party. In some embodiments, the machine learning model is a linear model providing a relationship between myopia progression and two or more features corresponding to the input data. In some embodiments, the machine learning model is generated using a machine learning procedure comprising multivariate linear regression analysis. In some embodiments, the machine learning model comprises a logistic regression model. In some embodiments, the machine learning model comprises a support vector machine model. In some embodiments, the prediction comprises a likelihood of progression to a higher degree of myopia. In some embodiments, the higher degree of myopia is low myopia, moderate myopia, or high myopia. In some embodiments, the low myopia corresponds to a spherical equivalent of −3.00 diopters or less. In some embodiments, the moderate myopia corresponds to a spherical equivalent between −3.00 diopters to −6.00 diopters. In some embodiments, the high myopia corresponds to a spherical equivalent of −6.00 diopters or more. In some embodiments, the individual has myopia. In some embodiments, the individual does not have myopia. In some embodiments, the prediction comprises an age of onset or time to onset of myopia in an individual who does not currently have myopia. In some embodiments, the prediction comprises a rate of progression of myopia. In some embodiments, the rate of progression comprises a predicted change in degree of myopia within a time period. In some embodiments, the time period is no more than about 6 months, about 1 year, about 2 years, about 3 years, about 4 years, about 5 years, about 6 years, about 7 years, about 8 years, about 9 years, or no more than about 10 years. In some embodiments, the predicted change in degree of myopia is about 1 diopters or less, from about 1 to about 2 diopters, from about 2 to about 3 diopters, from about 3 to about 4 diopters, from about 4 to about 5 diopters, from about 5 to about 6 diopters, from about 6 to about 7 diopters, from about 7 to about 8 diopters, from about 8 to about 9 diopters, from about 9 to about 10 diopters, from about 10 to about 12 diopters, from about 12 to about 15 diopters, from about 15 to about 20 diopters, from about 20 to about 30 diopters, from about 30 to about 40 diopters, or from about 40 to about 50 diopters. In some embodiments, the prediction comprises a predicted change in myopia for one or both eyes of the individual. In some embodiments, the data comprises an age and refraction, optionally under cycloplegia, of one or both eyes of the individual measured at one or more time points. In some embodiments, the one or more time points comprises at least 2, at least 3, at least 4, at least 5, at least 6, at least 7, at least 8, at least 9, or at least 10 time points. In some embodiments, the data is processed to calculate a spherical equivalent based on the refraction. In some embodiments, the prediction has an accuracy of at least 80% when tested against an independent data set of at least 200 samples. In some embodiments, a recommendation for treatment is provided to the individual or the third party. In some embodiments, a treatment is prescribed to the individual. In some embodiments, a treatment is provided to the individual. In some embodiments, the treatment comprises atropine eye drops. In some embodiments, the treatment comprises orthokeratology. In some embodiments, the treatment comprises atropine eye drops, multifocal contact lenses, orthokeratology, multifocal eyeglasses, or any combination thereof. In some embodiments, further testing is performed on the individual for one or more ophthalmic conditions or disorders associated with myopia. In some embodiments, the one or more ophthalmic conditions or disorders comprises cataract, glaucoma, myopic maculopathy, retinal detachment, macular edema, choroidal neovascularization, staphyloma, or any combination thereof. In some embodiments, the further testing comprises an optometry exam, genetic testing, imaging one or both eyes of the individual, screening for ocular disorders or conditions, or any combination thereof. In some embodiments, the individual is no more than 5 years old, 6 years old, 7 years old, 8 years old, 9 years old, 10 years old, 11 years old, 12 years old, 13 years old, 14 years old, 15 years old, 16 years old, 17 years old, 18 years old, 19 years old, 20 years old, 21 years old, 22 years old, 23 years old, 24 years old, 25 years old, 26 years old, 27 years old, 28 years old, 29 years old, or no more than 30 years old. In some embodiments, the individual is male or female. In some embodiments, the data comprises average exposure to sunlight or natural lighting, intensity of sunlight exposure, average UV index, latitude, or any combination thereof. In some embodiments, the input data is obtained from an eye examination. In some embodiments, the input data comprises age, gender, nationality, ethnicity, spherical equivalent (SE) measurement of one or both eyes, In some embodiments, the machine learning model is a classifier. In some embodiments, the machine learning model is a regression model.

In another aspect, disclosed herein is non-transitory computer readable medium including instructions executable by a processor to create an application comprising: a) a software module obtaining input data of the individual; b) a software module evaluating the input data using a machine learning model to generate a prediction of myopia onset or myopia progression, wherein the machine learning model has a sensitivity of at least about 90% and a specificity of at least about 90% when evaluated against an independent data set of at least 200 samples; and c) a software module providing the prediction to the individual or a third party. In some embodiments, the machine learning model is a linear model providing a relationship between myopia progression and two or more features corresponding to the input data. In some embodiments, the machine learning model is generated using a machine learning procedure comprising multivariate linear regression analysis. In some embodiments, the machine learning model comprises a logistic regression model. In some embodiments, the machine learning model comprises a support vector machine model. In some embodiments, the prediction comprises a likelihood of progression to a higher degree of myopia. In some embodiments, the higher degree of myopia is low myopia, moderate myopia, or high myopia. In some embodiments, the low myopia corresponds to a spherical equivalent of −3.00 diopters or less. In some embodiments, the moderate myopia corresponds to a spherical equivalent between −3.00 diopters to −6.00 diopters. In some embodiments, the high myopia corresponds to a spherical equivalent of −6.00 diopters or more. In some embodiments, the individual has myopia. In some embodiments, the individual does not have myopia. In some embodiments, the prediction comprises an age of onset or time to onset of myopia in an individual who does not currently have myopia. In some embodiments, the prediction comprises a rate of progression of myopia. In some embodiments, the rate of progression comprises a predicted change in degree of myopia within a time period. In some embodiments, the time period is no more than about 6 months, about 1 year, about 2 years, about 3 years, about 4 years, about 5 years, about 6 years, about 7 years, about 8 years, about 9 years, or no more than about 10 years. In some embodiments, the predicted change in degree of myopia is about 1 diopters or less, from about 1 to about 2 diopters, from about 2 to about 3 diopters, from about 3 to about 4 diopters, from about 4 to about 5 diopters, from about 5 to about 6 diopters, from about 6 to about 7 diopters, from about 7 to about 8 diopters, from about 8 to about 9 diopters, from about 9 to about 10 diopters, from about 10 to about 12 diopters, from about 12 to about 15 diopters, from about 15 to about 20 diopters, from about 20 to about 30 diopters, from about 30 to about 40 diopters, or from about 40 to about 50 diopters. In some embodiments, the prediction comprises a predicted change in myopia for one or both eyes of the individual. In some embodiments, the data comprises an age and refraction, optionally under cycloplegia, of one or both eyes of the individual measured at one or more time points. In some embodiments, the one or more time points comprises at least 2, at least 3, at least 4, at least 5, at least 6, at least 7, at least 8, at least 9, or at least 10 time points. In some embodiments, the data is processed to calculate a spherical equivalent based on the refraction. In some embodiments, the prediction has an accuracy of at least 80% when tested against an independent data set of at least 200 samples. In some embodiments, a recommendation for treatment is provided to the individual or the third party. In some embodiments, a treatment is prescribed to the individual. In some embodiments, a treatment is provided to the individual. In some embodiments, the treatment comprises atropine eye drops. In some embodiments, the treatment comprises orthokeratology. In some embodiments, the treatment comprises atropine eye drops, multifocal contact lenses, orthokeratology, multifocal eyeglasses, or any combination thereof. In some embodiments, further testing is performed on the individual for one or more ophthalmic conditions or disorders associated with myopia. In some embodiments, the one or more ophthalmic conditions or disorders comprises cataract, glaucoma, myopic maculopathy, retinal detachment, macular edema, choroidal neovascularization, staphyloma, or any combination thereof. In some embodiments, the further testing comprises an optometry exam, genetic testing, imaging one or both eyes of the individual, screening for ocular disorders or conditions, or any combination thereof. In some embodiments, the individual is no more than 5 years old, 6 years old, 7 years old, 8 years old, 9 years old, 10 years old, 11 years old, 12 years old, 13 years old, 14 years old, 15 years old, 16 years old, 17 years old, 18 years old, 19 years old, 20 years old, 21 years old, 22 years old, 23 years old, 24 years old, 25 years old, 26 years old, 27 years old, 28 years old, 29 years old, or no more than 30 years old. In some embodiments, the individual is male or female. In some embodiments, the data comprises average exposure to sunlight or natural lighting, intensity of sunlight exposure, average UV index, latitude, or any combination thereof. In some embodiments, the input data is obtained from an eye examination. In some embodiments, the input data comprises age, gender, nationality, ethnicity, spherical equivalent (SE) measurement of one or both eyes, In some embodiments, the machine learning model is a classifier. In some embodiments, the machine learning model is a regression model.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or applications file contains at least one drawing executed in color. Copies of this patent or patent application publication with color drawing(s) will be provided by the office upon request and payment of the necessary fee.

A better understanding of the features and advantages of the present invention will be obtained by reference to the following detailed description that sets forth illustrative embodiments, in which the principles of the invention are utilized, and the accompanying drawings of which:

FIG. 1A shows a flow chart for processing raw training data from Zhongshan Ophthalmic Centre for use in training machine learning models or classifiers described herein;

FIG. 1B shows a flow chart for processing raw training data from Shanghai Children Eye Study for use in training machine learning models or classifiers described herein;

FIG. 1C shows a flow chart for processing raw training data from Beijing Children Eye Study for use in training machine learning models or classifiers described herein;

FIG. 2A: Receiver Operating Curve (ROC) of high myopia classification via Logistic Regression model derived from Guangzhou Myopia Study. Accuracy: 82.1%; Sensitivity: 88.38%. Specificity: 82.61%; Binary classification between myopia (SE<0) classified as 0 and high myopia (SE≤−6.0) classified as 1.

FIG. 2B: Receiver Operating Curve (ROC) of high myopia classification via Logistic Regression model derived from Shanghai Children Myopia Study. Accuracy:

90.26%; Sensitivity 89.86%; Specificity 90.27%. Binary classification between myopia (SE<0) classified as 0 and high myopia (SE≤−6.0) classified as 1.

FIG. 3 shows receiver operating curve (ROC) for the Beijing Tongren Eye Center Children's Study;

FIG. 4 shows the receiver operating curve (ROC) for the Shanghai Children Eye Study;

FIG. 5 shows the receiver operating curve (ROC) for another study;

FIG. 6 shows a comparison of Predictive SE progression (red) vs SE Progression using GOV lens (blue) for a plurality of samples. The y-axis denotes the power increase in diopters; the x-axis denotes the sample IDs used in the model. For each sample ID, two points (one red, one blue) are plotted in the graphic. The mean predicted power increase was −2.02 D. The mean of power increase when using GOV lens was −0.54 D

FIG. 7 shows a comparison of Predictive SE progression (red) vs SE Progression using GOV lens (blue) displaying myopia power increase over time. The y-axis denotes the power increase in diopters; the x-axis donates progression in years. The mean predicted power increase was −2.02 D. The mean of power increase when using GOV lens was −0.54 D. A Welch two sample independent/unpaired t-test was conducted. The results proved significant (p-value=2.2×10\hat{ }(−16)).

FIG. 8 shows a comparison of outcomes with and without use of GOV lens. The mean predicted power increase was −2.03 D. The mean of power increase when using GOV lens was −0.55 D. A Welch two sample independent/unpaired t-test was conducted. The results proved significant (p-value=2.2×10\hat{ }(−16)).

FIG. 9A shows the accuracy of predictions generated with a linear regression model with 10× cross-validation on data from Zhongshan Ophthalmic Centre;

FIG. 9B shows the accuracy of predictions generated with a logistic regression model on data from Zhongshan Ophthalmic Centre;

FIG. 9C shows the accuracy of predictions generated with a support vector machine (SVM) on data from Zhongshan Ophthalmic Centre;

FIG. 9D shows the accuracy of predictions generated with a logistic regression model on data from the Beijing Children's Tongren Eye Study; and

FIG. 9E shows the accuracy of predictions generated with a support vector machine (SVM) model on data from the Beijing Children's Tongren Eye Study.

FIG. 10 shows a comparison of Predictive SE progression (red) vs SE Progression using GOV lens (blue). The y-axis denotes the power increase in diopters; the x-axis denotes the sample IDs used in the model. For each sample ID, two points (one red, one blue) are plotted in the graphic. The mean predicted power increase was −2.04 D. The mean of power increase when using GOV lens was −0.41 D.

FIG. 11 shows a comparison of outcomes with and without use of GOV lens. The mean of power increase when using GOV lens was −0.41 D. The mean predicted power increase was −2.04 D. A Welch two-sample independent/unpaired t-test was conducted. The results proved significant with p-value=2.2×10\hat{ }(−16).

FIG. 12 shows myopia progression over time. Spherical equivalent (SE) over age as seen in Guangzhou Myopia Study (green) and Beijing Undergraduate Students Study (blue). The X-axis denotes 2-20 years of age. The Y-axis denotes SE with median, 25th and 75th percentiles and 95% confidence intervals.

FIGS. 13A-13F show scatter plots of linear regression model and histograms of prediction error of Guangzhou Myopia Study (GMS) internal validation set, Study 1 and Study 2. FIG. 13A) GMS R²=0.964; FIG. 13B) GMS prediction error within 1 D=86%; FIG. 13C) Study 1 R²=0.950; FIG. 13D) Study 1 prediction error within 1 D=83%; FIG. 13E) Study 2 R²=0.950; FIG. 13F) Study 2 prediction error within 1 D=83%.

FIGS. 14A-14F show scatter plots of linear regression model and histograms of prediction error of Shanghai Children Myopia Study (SCMS), Beijing Children Eye Study (BCES) and San Diego Children Study (SDCS). FIG. 14A) SCMS R²=0.807; FIG. 14B) SCMS prediction error within 1 D=86%; FIG. 14C) BCES R²=0.749; FIG. 14D) BCES prediction error within 1 D=74%; FIG. 14E) SDCS R²=0.891; FIG. 14F) SDCS prediction error within 1 D=87%.

FIGS. 15A-15F show normalized confusion matrices and receiver operating curve (ROC) of high myopia classification via logistic regression model for GSM internal validation. FIG. 15A) GMS internal validation: accuracy=94.31%, sensitivity=98.96%, specificity=93.70%; FIG. 15B) GMS internal validation area under ROC (AUROC)=0.99; FIG. 15C) Study 1: accuracy=93.95%, sensitivity=96.28%, specificity=93.62%; FIG. 15D) Study 1 AUROC=0.99; FIG. 15E) Study 2: accuracy=93.93%, sensitivity=96.29%, specificity=93.59%; FIG. 15F) Study 2 AUROC=0.99.

FIGS. 16A-16F show normalized confusion matrices and receiver operating curve (ROC) of high myopia classification via logistic regression model for SCMS, BCES and SDCS. FIG. 16A) SCMS: accuracy=89.54%, sensitivity=90.91%, specificity=89.49%; FIG. 16B) SCMS AUROC=0.96; FIG. 16C) BCES: accuracy=96.18%, sensitivity=90.00%, specificity=96.27%; FIG. 16D) BCES AUROC=0.99; FIG. 16E) SDCS: accuracy=89.54%, sensitivity=90.91%, specificity=89.49%; FIG. 16F) SDCS AUROC=0.98.

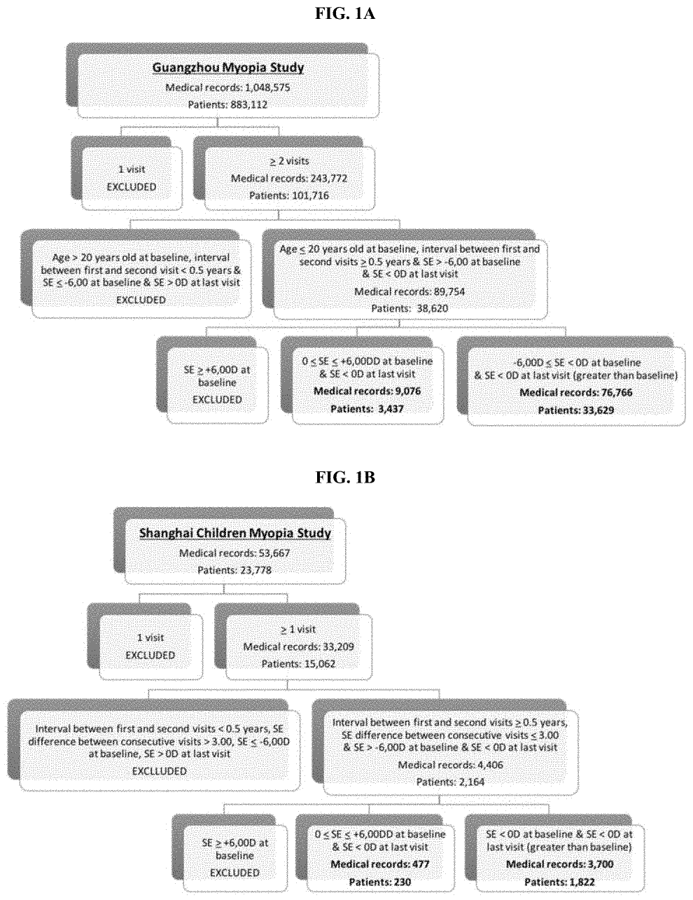

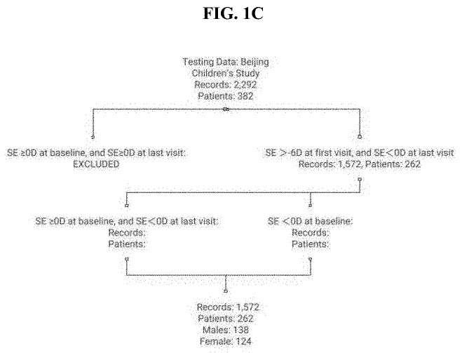

### DETAILED DESCRIPTION OF THE DISCLOSURE

It is recognized that implementation of machine learning algorithms providing accurate predictions of myopia onset and/or progression can be achieved through one or combinations of technical features of the present disclosure. According to some aspects, disclosed herein is a diagnostic tool to correctly identify eye-related issues by presenting a machine learning framework developed for ophthalmic diseases or conditions such as myopia onset and myopia progression. This framework can be applied towards medical data or information about the individual such as age, refraction

In some embodiments, the algorithm(s) disclosed herein are implemented on various computing devices. The computing devices include portable communication devices such as smart phones, tablets, desktop computers, laptops, and other computing devices.

In some embodiments, disclosed herein is a computer-implemented method for predicting myopia progression in an individual, comprising: a) obtaining data of the individual; b) evaluating the data using a machine learning algorithm to generate a prediction of myopia progression; and c) providing the prediction to the individual or a third party.

In some embodiments, disclosed herein is a computer-implemented system comprising: a) an electronic device comprising: a processor, a memory, and an operating system configured to perform executable instructions; and b) a computer program stored in the memory of the electronic device, the computer program including instructions executable by the user electronic device to create an application comprising: i) a software module obtaining data of the individual; ii) a software module evaluating the data using a machine learning algorithm to generate a prediction of myopia progression; and iii) a software module providing the prediction to the individual or a third party.

In another aspect, disclosed herein is non-transitory computer readable medium including instructions executable by a processor to create an application comprising: a) a software module obtaining data of the individual; b) a software module evaluating the data using a machine learning algorithm to generate a prediction of myopia progression; and c) a software module providing the prediction to the individual or a third party.

In another aspect, disclosed herein is a method for performing a personalized evaluation of an individual for myopia onset or myopia progression, comprising: a) obtaining input data of the individual; b) evaluating the input data using a machine learning model to generate a prediction of myopia onset or myopia progression, wherein the machine learning model has a sensitivity of at least about 90% and a specificity of at least about 90% when evaluated against an independent data set of at least 200 samples; and c) providing the prediction to the individual or a third party.

In another aspect, disclosed herein is a computer-implemented system comprising: c) an electronic device comprising: a processor, a memory, and an operating system configured to perform executable instructions; and d) a computer program stored in the memory of the electronic device, the computer program including instructions executable by the user electronic device to create an application comprising: i) a software module obtaining input data of the individual; ii) a software module evaluating the input data using a machine learning model to generate a prediction of myopia onset or myopia progression, wherein the machine learning model has a sensitivity of at least about 90% and a specificity of at least about 90% when evaluated against an independent data set of at least 200 samples; and iii) a software module providing the prediction to the individual or a third party.

In another aspect, disclosed herein is non-transitory computer readable medium including instructions executable by a processor to create an application comprising: a) a software module obtaining input data of the individual; b) a software module evaluating the input data using a machine learning model to generate a prediction of myopia onset or myopia progression, wherein the machine learning model has a sensitivity of at least about 90% and a specificity of at least about 90% when evaluated against an independent data set of at least 200 samples; and c) a software module providing the prediction to the individual or a third party.

### AI Diagnostics

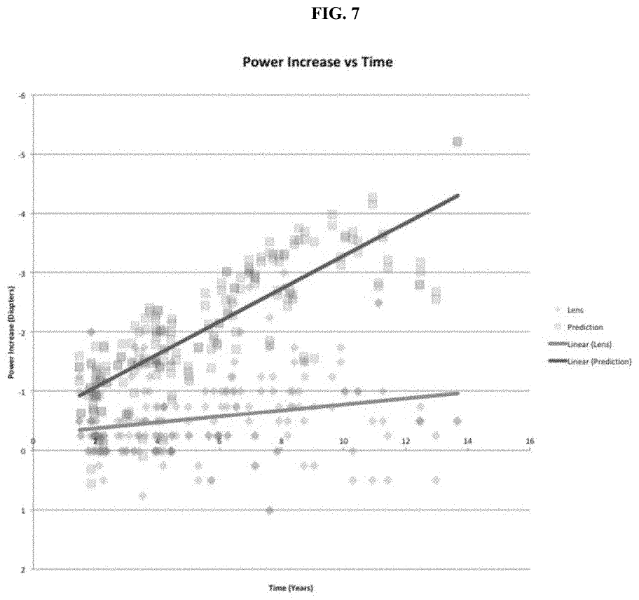

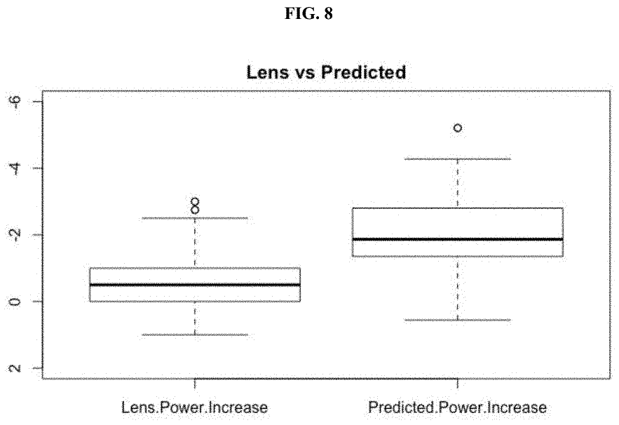

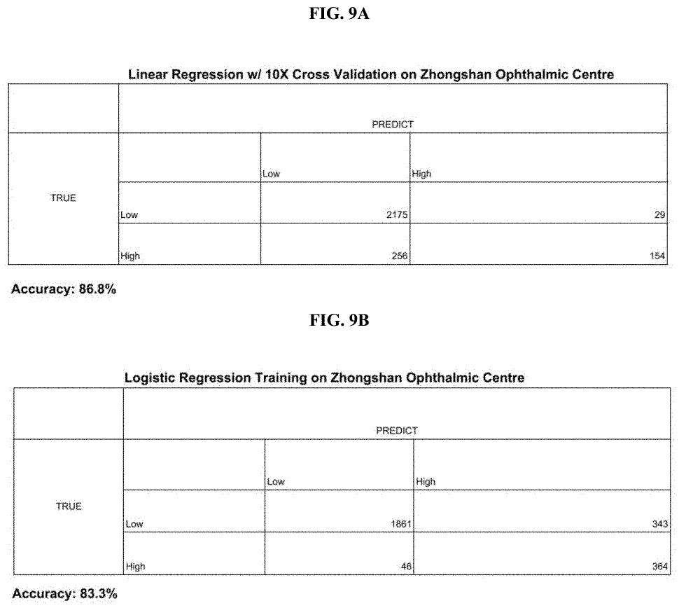

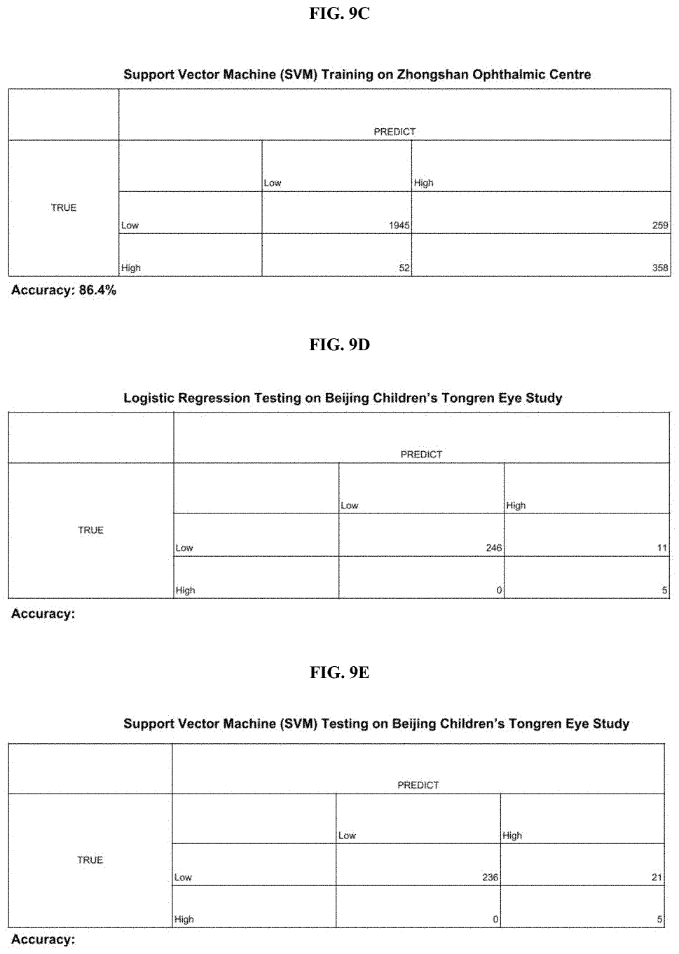

In some aspects, disclosed herein are artificial intelligence-based diagnostics for predicting the onset and/or progression of myopia. In some embodiments, the AI diagnostic comprises machine learning algorithms. In some embodiments, machine learning algorithms are implemented across one or more cohorts. In some embodiments, machine learning algorithms are implemented in in or more cohorts to predict the onset of high myopia and/or myopia progression in children and adults (e.g., school children and adults in Asia). In some embodiments, a discovery cohort of patients is used for algorithm training validation. In some embodiments, external validation was confirmed through the use of two separate independent cohorts. In some embodiments, prediction accuracy is assessed via area under the receiver operating characteristics curve (AUC). In some embodiments, records of individuals in hospital-based databases and follow-up records of participants in population-based cohorts are included in the data. In some embodiments, the machine learning algorithm accurately predicts the presence of high myopia for external validations of one or more cohorts. In some embodiments, the algorithm is validated using at least 2 cohorts. In one embodiment, with respect to the prediction of high myopia by 20 years of age as a surrogate of high myopia in adulthood, the algorithm provides clinically meaningful predictions over a period of years. In addition, intervention by atropine or OrthoK therapy can significantly reduce progression. The algorithms in the present disclosure provide an innovative new avenue for prediction and reduction of myopia progression. In some embodiments, the algorithm predicts a power increase over time (e.g., in diopters). In some embodiments, the algorithm predicts a power increase over time for two or more treatments (or lack thereof). For example, the algorithm can generate a first prediction of a first power increase over time for no treatment and a second prediction of a second power increase over time for a particular treatment. In some embodiments, a comparison of the two or more predictions is provided. For example, FIG. 7 provides an illustrative example of a graph showing power increase over time for GOV lens compared to without the lens. In some embodiments, a comparison of the predictions can be displayed or otherwise made available to a user or a healthcare provider or patient. This allows the person to make an informed decision regarding treatment options. For example, when a particular treatment (e.g., atropine drops or OrthoK therapy) is predicted to reduce power increase by 1 diopter over the next 3 year period, a person may act on that information to choose the treatment. In some embodiments, the systems and methods disclosed herein generate a recommendation based on the prediction(s). In some embodiments, the methods disclosed herein comprise a treatment administration step based on the prediction(s). The treatment can be a particular treatment specifically directed to treating, preventing, or reducing myopia and/or myopia related symptoms or conditions.

### Machine Learning

Disclosed herein, in various embodiments, are machine learning methods for analyzing user data including, for example, eye-related data. In some embodiments, the machine learning algorithms disclosed herein are used to train models or classifiers that generate predictions of myopia onset and/or myopia progression. In some embodiments, the model or classifier is validated using a test data set which uses data not used in the training of the algorithm. Accordingly, the algorithms and models disclosed herein can provide clinically meaningful predictions of refractive error over various time spans, while accurately predicting the onset of high myopia in internal and external validations. Improved predictive models can help identify high-risk patients who would benefit from earlier preventive interventions. The systems and methods disclosed herein offer a new avenue for prediction of visual outcomes in populations with high myopia prevalence while identifying individuals at risk for developing high myopia, potentially mitigating vision loss while combating both economic and healthcare burdens. In some embodiments, the algorithms and models disclosed herein are tailored to specific population cohorts. For example, a machine learning algorithm or model may be trained using training data corresponding to a specific population delineated by ethnicity, location, or other common factor (e.g., ethnic China, Hispanic, European, etc). The resulting trained model can generate predictions that are more accurate for an individual belonging to that specific population than a model trained using a mixed population training set.

In some embodiments, the algorithms and models disclosed herein provide clinically meaning and/or accurate predictions of myopia onset and/or progression over a time span. In some embodiments, the time span is about 4 years to about 20 years. In some embodiments, the time span refers to the age of an individual. For example, a time span of about 4 to about 20 years can refer to an age of about 4 to about 20 years old. In some embodiments, the time span is about 4 years to about 6 years, about 4 years to about 8 years, about 4 years to about 10 years, about 4 years to about 12 years, about 4 years to about 14 years, about 4 years to about 16 years, about 4 years to about 18 years, about 4 years to about 20 years, about 6 years to about 8 years, about 6 years to about 10 years, about 6 years to about 12 years, about 6 years to about 14 years, about 6 years to about 16 years, about 6 years to about 18 years, about 6 years to about 20 years, about 8 years to about 10 years, about 8 years to about 12 years, about 8 years to about 14 years, about 8 years to about 16 years, about 8 years to about 18 years, about 8 years to about 20 years, about 10 years to about 12 years, about 10 years to about 14 years, about 10 years to about 16 years, about 10 years to about 18 years, about 10 years to about 20 years, about 12 years to about 14 years, about 12 years to about 16 years, about 12 years to about 18 years, about 12 years to about 20 years, about 14 years to about 16 years, about 14 years to about 18 years, about 14 years to about 20 years, about 16 years to about 18 years, about 16 years to about 20 years, or about 18 years to about 20 years. In some embodiments, the time span is about 4 years, about 6 years, about 8 years, about 10 years, about 12 years, about 14 years, about 16 years, about 18 years, or about 20 years. In some embodiments, the time span is at least about 4 years, about 6 years, about 8 years, about 10 years, about 12 years, about 14 years, about 16 years, or about 18 years. In some embodiments, the time span is at most about 6 years, about 8 years, about 10 years, about 12 years, about 14 years, about 16 years, about 18 years, or about 20 years.

In some embodiments, the machine learning algorithm and/or classifier described herein exhibits performance metrics such as accuracy, sensitivity, specificity, positive predictive value, negative predictive value, and/or AUC for an independent sample set. In some embodiments, the classifier exhibits performance metrics such as higher accuracy, sensitivity, specificity, positive predictive value, negative predictive value, and/or AUC for an independent sample set compared to an average human clinician (e.g. an average ophthalmologist). In some embodiments, the classifier provides an accuracy of at least 70%, at least 75%, at least 80%, at least 85%, at least 90%, at least 91%, at least 92%, at least 93%, at least 94%, at least 95%, at least 96%, at least 97%, at least 98%, or at least 99% when tested against at least 100, 200, 300, 400, 500, 600, 700, 800, 900, or 1000 independent samples. In some embodiments, the classifier provides a sensitivity (true positive rate) of at least 70%, at least 75%, at least 80%, at least 85%, at least 90%, at least 91%, at least 92%, at least 93%, at least 94%, at least 95%, at least 96%, at least 97%, at least 98%, at least 99%, and/or a specificity (true negative rate) of at least 70%, at least 75%, at least 80%, at least 85%, at least 90%, at least 91%, at least 92%, at least 93%, at least 94%, at least 95%, at least 96%, at least 97%, at least 98%, or at least 99% when tested against at least 100, 200, 300, 400, 500, 600, 700, 800, 900, or 1000 independent samples. In some embodiments, the classifier provides a positive predictive value (PPV) of at least 70%, at least 75%, at least 80%, at least 85%, at least 90%, at least 91%, at least 92%, at least 93%, at least 94%, at least 95%, at least 96%, at least 97%, at least 98%, or at least 99% when tested against at least 100, 200, 300, 400, 500, 600, 700, 800, 900, or 1000 independent samples. In some embodiments, the classifier provides a negative predictive value (NPV) of at least 70%, at least 75%, at least 80%, at least 85%, at least 90%, at least 91%, at least 92%, at least 93%, at least 94%, at least 95%, at least 96%, at least 97%, at least 98%, or at least 99% when tested against at least 100, 200, 300, 400, 500, 600, 700, 800, 900, or 1000 independent samples. In some embodiments, the classifier has an area under the curve (AUC) of at least 0.7, 0.75, 0.8, 0.85, 0.9, 0.91, 0.92, 0.93, 0.94, 0.95, 0.96, 0.97, 0.98 or 0.99 when tested against at least 100, 200, 300, 400, 500, 600, 700, 800, 900, or 1000 or more independent samples. In some embodiments, the classifier has a weighted error compared to one or more independent experts of no more than 20%, no more than 15%, no more than 12%, no more than 10%, no more than 9%, no more than 8%, no more than 7%, no more than 6%, no more than 5%, no more than 4%, no more than 3%, no more than 2%, or no more than 1% when tested against at least 100, 200, 300, 400, 500, 600, 700, 800, 900, or 1000 independent samples. The samples can refer to medical records or data for individuals comprising information for the features utilized by the classifier.

Various algorithms can be used to generate models that generate a prediction based on the data analysis. In some instances, machine learning methods are applied to the generation of such models (e.g. trained classifier). In some embodiments, the model is generated by providing a machine learning algorithm with training data in which the expected output is known in advance.

In some embodiments, the systems, devices, and methods described herein generate one or more recommendations such as treatment and/or healthcare options for a subject. In some embodiments, the systems, devices, and methods herein comprise a software module providing one or more recommendations to a user. In some embodiments, the treatment and/or healthcare option are specific to the diagnosed disease or condition. For example, a recommendation can suggest a nearby hospital, doctor, or clinic with the requisite facilities or resources for treating the myopia or myopia-related condition(s).

In some embodiments, a classifier or trained machine learning algorithm of the present disclosure comprises a feature space. In some cases, the classifier comprises two or more feature spaces. The two or more feature spaces may be distinct from one another. In some embodiments, a feature space comprises information such as age and a corresponding refraction or refractive error for measured for one or both eyes of an individual. When training the machine learning algorithm, training data is input into the algorithm which processes the input features to generate a model. In some embodiments, the machine learning algorithm is provided with training data that includes the classification (e.g. diagnostic or test result), thus enabling the algorithm to train by comparing its output with the actual output to modify and improve the model. In some embodiments, the classification is the myopic progression such as rate of progression, amount of progression, age of onset of progression, or other suitable metric for assessing myopia and its progression. This is often referred to as supervised learning. Alternatively, in some embodiments, the machine learning algorithm can be provided with unlabeled or unclassified data, which leaves the algorithm to identify hidden structure amongst the cases (referred to as unsupervised learning). Sometimes, unsupervised learning is useful for identifying the features that are most useful for classifying raw data into separate cohorts.

In some embodiments, one or more sets of training data are used to train a machine learning algorithm. Although exemplar embodiments of the present disclosure include machine learning algorithms that use convolutional neural networks, various types of algorithms are contemplated. In some embodiments, the algorithm utilizes a predictive model such as a neural network, a decision tree, a support vector machine, or other applicable model. In some embodiments, the machine learning algorithm is selected from the group consisting of a supervised, semi-supervised and unsupervised learning, such as, for example, a support vector machine (SVM), a Naïve Bayes classification, a random forest, an artificial neural network, a decision tree, a K-means, learning vector quantization (LVQ), self-organizing map (SOM), graphical model, regression algorithm (e.g. linear, logistic, multivariate, association rule learning, deep learning, dimensionality reduction and ensemble selection algorithms. In some embodiments, the machine learning algorithm is selected from the group consisting of: a support vector machine (SVM), a Naïve Bayes classification, a random forest, and an artificial neural network. Machine learning techniques include bagging procedures, boosting procedures, random forest algorithms, and combinations thereof. Illustrative algorithms for analyzing the data include but are not limited to methods that handle large numbers of variables directly such as statistical methods and methods based on machine learning techniques. Statistical methods include penalized logistic regression, prediction analysis of microarrays (PAM), methods based on shrunken centroids, support vector machine analysis, and regularized linear discriminant analysis.

### Methods of Treatment

Disclosed herein are systems and methods for detecting myopia onset and/or progression using artificial intelligence that allow for treating or preventing myopia onset and/or progression. The detection or prediction of myopia onset or myopia progression allows for various treatment or prevention modalities to be explored. In some embodiments, following the detection of myopia onset, one or more treatments are recommended or provided. In some embodiments, the systems and methods disclosed herein comprise steps for treating or preventing myopia onset and/or progression. In some embodiments, the treatment and/or prevention is personalized based on the associated prediction or evaluation of myopia onset and/or progression. For example, given a prediction of myopia onset when myopia has not yet occurred or when myopia is in the early stages, a recommended prevention step may be lifestyle changes such as increasing outdoor activities or exposure to sunlight, or reducing near-work such as reading. For example, a prevention step may be to recommend exposure to sunlight for at least 1, 2, 3, 4, 5, or 6 hours for at least 1, 2, 3, 4, 5, 6, or 7 days a week. In another example, given a prediction of myopia progression, a recommended step for preventing or reducing myopia progression can be the use of atropine eye drops. In some embodiments, the prediction is for a likelihood of high myopia such as a spherical equivalent of −6.00 diopters or more. In the case of high myopia, which is associated with more serious ophthalmic conditions or disorders, the treatment can include topical atropine (e.g., atropine eye drops) or orthokeratology (OK) lenses. In some embodiments, when high myopia is not predicted (e.g., the individual is classified into a lower risk category), the recommendation is provided for prevention (e.g., lifestyle changes).

### Diagnostic Platforms, Systems, Devices, and Media

Provided herein, in certain aspects, are platforms, systems, devices, and media for analyzing medical data according to any of the methods of the present disclosure. In some embodiments, the systems and electronic devices are integrated with a program including instructions executable by a processor to carry out analysis of medical data. In some embodiments, the analysis is performed locally on the device utilizing local software integrated into the device. In some embodiments, the analysis is performed remotely on a remote system or server. In some embodiments, the analysis is performed remotely on the cloud after the data is uploaded by the system or device over a network. In some embodiments, the system or device is an existing system or device adapted to interface with a web application operating on the network or cloud for uploading and analyzing user data. In some embodiments, the system or device provides for portable data storage such as on a USB drive or other portable hard drive. Portable storage enables the data to be transferred to a device capable of performing analysis on the data and/or which has network connectivity for uploading the data for remote analysis on the cloud.

In some embodiments, the platforms, systems, devices, and media disclosed herein provide interfaces or portals for receiving user or patient data. In some embodiments, the data is uploaded by a user (e.g., a user via a smartphone app) or a healthcare provider (e.g., an optometrist or ophthalmologist). In some embodiments, the data is uploaded manually such as by typing or entering information into a window or other prompt. In some embodiments, the data is uploaded via a file, for example, a medical history file. In some embodiments, the file is formatted and/or converted into an appropriate format or mined to obtain relevant information such as vision measurements (e.g., diopters required to correct myopia).

### Cloud-Based Diagnosis

Provided herein, in certain embodiments, are systems, devices, and methods for providing a web application or portal for remote data analysis or diagnosis (e.g., “cloud” diagnosis). In order to tackle the reproducibility and transparency issues brought on by training and testing on a protected or proprietary dataset provided herein is an easy-to-use application (e.g. web tool) that allows testing of a model on any provided user data. In some embodiments, the application allows a user to load a trained model and predicts the diagnosis of a user based on associated or provided user data.

In some embodiments, the electronic device comprises a portal providing one or more tools for a user to input user data or information such as name, address, email, phone number, and/or other identifying information. In some embodiments, the portal comprises an interface for obtaining or entering medical data such as, for example, a medical history relating to myopia or other eye exam data. In some embodiments, the portal is configured to receive medical data for use in the prediction or diagnosis from device through a network (e.g. receives medical data provided by a user smartphone through the internet via a mobile app or web portal).

In some embodiments, the portal provides the user with the option to receive the results of the analysis by email, messaging (e.g. SMS, text message), physical printout (e.g. a printed report), social media, by phone (e.g. an automated phone message or a consultation by a healthcare provider or adviser), or a combination thereof. In some embodiments, the prediction is provided to the user. In some embodiments, the portal is displayed on a digital screen of the electronic device. In some embodiments, the electronic device comprises an analog interface. In some embodiments, the electronic device comprises a digital interface such as a touchscreen.

### Digital Processing Device

In some embodiments, the systems, devices, platforms, media, methods and applications described herein include a digital processing device, a processor, or use of the same. For example, in some embodiments, the digital processing device is part of a point-of-care device such as a medical diagnostic device integrating the diagnostic software described herein. In some embodiments, the medical diagnostic device is a consumer-facing portable medical diagnostic device configured for use outside of the clinical setting (e.g. consumer use at home such as on a smart phone app). In further embodiments, the digital processing device includes one or more processors or hardware central processing units (CPU) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device. In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In some embodiments, the non-volatile memory comprises magnetoresistive random-access memory (MRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a subject. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In some embodiments, the display is E-paper or E ink. In other embodiments, the display is a video projector. In still further embodiments, the display is a combination of devices such as those disclosed herein.

In some embodiments, the digital processing device includes an input device to receive information from a subject. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Non-Transitory Computer Readable Storage Medium

In some embodiments, the platforms, media, methods and applications described herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, media, methods and applications described herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™ JavaServer Pages (JSP), Hypertext Preprocessor (PHP), Python™, Ruby, Tcl, Smalltalk, WebDNA, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile digital processing device such as a smartphone. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g. not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

In some embodiments, the platforms, media, methods and applications described herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of barcode, route, parcel, subject, or network information. In various embodiments, suitable databases include, by way of non-limiting examples, relational databases, non-relational databases, object oriented databases, object databases, entity-relationship model databases, associative databases, and XML, databases. In some embodiments, a database is internet-based. In further embodiments, a database is web-based. In still further embodiments, a database is cloud computing-based. In other embodiments, a database is based on one or more local computer storage devices.

### EXAMPLES

### Example 1

Due to new machine learning algorithms and its incorporation into the medical field, Artificial Intelligence (AI) has the potential to revolutionize disease diagnosis and prediction, leading to an overall higher standard of care. With the exponential incline of myopic cases within the last decade, a large repository of data has become available to predict the onset of high myopia at specific future time points. In doing so, preventative care can be enforced prior to the progression of myopic disease, therefore combating the current myopia epidemic, while lowering both economic and health burdens. Herein, both regression and classification machine learning models are implemented on several ophthalmologic cohorts. The rate of myopic progression is then compared to those who either used Atropine or wore orthokeratology (OK) lenses over a period of time. The model accurately predicts myopia progression and high myopia onset and shows any differences in progression rates between patients who sought interventive methods such as OK lens, and atropine, respectively. Through predicting myopia progression, the current rapid prevalence rates can be dampened by interventive techniques.

Methods

Data Collection and Cleaning

Data was composed primarily of patient's age and refraction under cycloplegia over various periods of time. GMS data had the most amount of information, consisting in children and adults with exam of both eyes and visits interval ranging from 0.5 to 6.12 years between each other. SCMS data consisted in children with exam of both eyes with mainly one follow-up visit after baseline. BCES cohort was composed of primary school-aged children with six annual follow-ups from 2011 to 2016 and refraction was measured for the right eye at each visit.

The data was first processed by calculating the spherical equivalent (SE) from refractive error, defined by the equation:

Spherical Equivalent=Spherical Diopter+½ (Cylindrical Diopter)

The annual progression of refractive error was calculated for each visit using the equation below:

Progression=(SE_baseline−SE)/(t_baseline−t)

The machine learning algorithms were trained on the extensive amount of data provided in GMS. The raw data was curated to only include patients with at least two visits; patients younger than 20 years old at the baseline visit; patients with follow-up periods at least six months apart; patients with at a baseline SE between +6,00 D and 0 D with final SE<0 D and a baseline SE between 0 D and −6,00 D with endpoint SE<0 D greater than baseline to denote myopic progression. For the SCMS dataset, an inclusion criteria of SE difference between consecutive visits 3.00 was also implemented.

After data cleaning, the GMS cohort consisted of 37,132 patients (20,306 males and 16,826 females) and a total of 86,040 medical records were implemented under separate machine learning algorithms; SCMS cohort consisted of 2,052 patients (1,093 males and 959 females) from a total of 4,177 medical records; and BCES cohort consisted of 1,572 medical records from 262 patients. A clear representation of the data cleansing process and inclusion/exclusion criteria can be seen in FIG. 12.

Applications of Regression and Classification Algorithms, Training and Validation

Machine learning often implores two distinct learning applications: regression or classification. Regression can be used to predict continuous value outputs, such as the degree of SE in myopia progression; whereas classification can be utilized to differentiate discrete outputs, such as the variance between myopia and high myopia. Multivariate linear regression analysis is a form of regression that creates a model to discern a linear relationship between two or more explanatory variables. This model was applied to the data to predict myopia progression by exploring the linear relationship between the following features: age at baseline visit, SE at baseline visit, time interval between baseline and follow-up visits and annual SE progression from first to second visit.

Logistic regression models is a form of classification-based application and was used to classify the likelihood of progression to high myopia, defined as an SE<−6,00 D. In implementing this form of machine learning, progression can be accurately predicted, and patients can be forewarned of subsequent high myopia.

Left and right eyes were used as independent training samples. GMS data was used for training and internal validation. The first two time points were used for training measures; subsequent time points were used for testing purposes. Patients were split into a 90:10 training-to-testing ratio and simultaneously evaluated using 10-fold cross-validation. External validation confirmed progression algorithms in other Chinese cohorts.

The metrics used to evaluate the multivariate linear regression model for both GMS internal validation and SCM external validation cohorts were mean absolute error (MAE), mean squared error (MSE) and root mean squared error (rMSE). MAE is calculated by taking of absolute errors, and is better applied when needing to distribute equal weight across all errors. The MSE equation is defined as the variance of the errors plus the square of the mean error MSE=(VAR(E)+(ME)\hat{ }2). rMSE is calculated by taking the square root of the MSE, and is particularly helpful when trying to weigh large errors. To evaluate the efficacy of logistic regression as a classification model of predicting progression to high myopia, the following metrics were used: accuracy, sensitivity of predicting high myopia, specificity of identifying non-progressors, and area under the ROC curve (AUC).

Intervention Studies

Myopia Control Study (MCS)

In order to evaluate the potential beneficial use of wearing Global OK-Vision (or GOV) lenses to control myopia progression, a retrospective study with 204 eyes of 102 myopic children was conducted. After data cleaning to exclude cases with less than 6.5 years of follow-up, the MCS dataset consisted of 136 eyes of 68 patients. Baseline data was defined as age and refraction under cycloplegia collected previous to initial GOV lens use, and patients were followed for at least two visits, with visits interval ranging from 1.49 to 6.46 years. SE was measured at baseline and power increase, defined as the difference of SE between that visit and baseline, was measured at each following visit. Baseline SE ranged from −0.50 D to −9.625 D.

The linear regression model trained using GMS dataset was applied to predict the expected myopic increase in this cohort as if they had not been using GOV. The ability of GOV to slow myopic progression was examined by comparing the actual power increase with the predicted power increase for each patient using a Welch's two-sample independent t-test.

Atropine Study

A study is conducted examining myopic progression in patients treated with atropine.

Results

Patients Characteristics

Table 1 contains a summary of the study population. In total, a number of medical records of patients from three different Chinese cohorts were used.

Guangzhou Myopia Study

The mean age at the time of baseline was 10.34 years old (STD: 3.5 years). The mean age at time of conclusion was 12.32 years old (STD: 3.66 years). The mean SE of right and left eyes at baseline were −2.31 and −2.36 respectively (STD: 1.75 right; 1.79 left). The mean SE of right and left eyes at the last exam were −3.80 and −3.88 respectively (STD: 1.78 right; 1.83 left). The mean time period between the first and second visits was 1.94 years (STD: 1.1 years) (Table 1). Training on linear regression with 10 fold cross validation produced mean MSE of 1.483 (STD: 0.114), mean rMSE of 1.217 (STD: 0.047) and MAE of 0.875 (STD: 0.023). Logistic regression produced mean accuracy of 82.10% (STD: 1.00), with sensitivity of 88.38% and specificity of 82.61%.

TABLE 1
General characteristics.

| | | GMS | SCMS | BCES | MCS |
|---|---|---|---|---|---|
| Raw data | Medical records (n) | 1,048,575 | 53,667 | 2,292 | 204 |
| | Patients (n) | 883,112 | 23,778 | 382 | 102 |
| Clean data | Medical records (n) | 86,040 | 4,177 | 1,572 | 136 |
| | Patients (n) | 37,132 | 2,052 | 262 | 68 |
| | Female (n/%) | 16,826/45.31 | 959/46.73 | 124/47.33 | — |
| | Follow-up, | 1.94 ± 1.1 | 0.73 ± 0.21 | 6 | 3.62 ± 1.48 |
| | mean ± STD (y) | | | | |
| | Age at first visit, | 10.34 ± 3.5 | 7.93 ± 3.2 | 6.34 ± 0.5 | 10.54 ± 2.75 |
| | mean ± STD (y) | | | | |
| | SE OD at first visit, | −2.31 ± 1.75 | −2.04 ± 1.82 | −0.14 ± 0.86 | −3.61 ± 1.89* |
| | mean ± STD (D) | | | | |
| | SE OS at first visit, | −2.36 ± 1.79 | −1.96 ± 1.8 | — | |
| | mean ± STD (D) | | | | |
| | Age at last visit, | 12.32 ± 3.66 | 8.66 ± 3.2 | −2.68 ± 1.87 | 16.15 ± 4.39 |
| | mean ± STD (y) | | | | |
| | SE OD at last visit, | −3.80 ± 1.78 | −2.85 ± 1.69 | −2.62 ± 1.8 | −0.41 ± 0.51* |
| | mean ± STD (D) | | | | |
| | SE OS at last visit, | −3.88 ± 1.83 | −2.77 ± 1.66 | — | |
| | mean ± STD (D) | | | | |

GMS = Guangzhou Myopia Study; SCMS = Shanghai Children Myopia Study. BCES = Beijing Children Eye Study; MCS = Myopia Control Study; n = number; STD = standard deviation; y = years; D = diopters;
*SE not calculated for each eye individually.

Shanghai Children Eye Study

The mean age at baseline was 7.93 years (STD: 3.2 years). The mean age at time of conclusion was 8.66 years old (STD: 3.2 years). The mean SE of right and left eyes at baseline were −2.04 and −1.96 respectively (STD: 1.82 right; 1.80 left). The mean SE of right and left eyes at the last exam were −2.85 and −2.77 respectively (STD: 1.69 right; 1.66 left). The mean time period between the first and last visits was 0.73 years (STD: 0.21 years) (Table 1).

Test on linear regression produced MSE of 0.45, rMSE of 0.67 and MAE of 0.53. Logistic regression produced accuracy of 90.26%, sensitivity of 89.86% and specificity of 90.27%.

Beijing Children Eye Study

The mean age at baseline was 6.34 (STD: 0.5 years). The mean age at time of conclusion was 11.34 years old (STD: 0.5 years). The mean SE of right eyes at baseline were −0.14 D (STD: 0.86 D) right. The mean SE of right eyes at the last exam were −1.57 D (STD: 1.92 D). The mean time period between the first and last visits was six years (Table 1).

Test on logistic regression and SVM produced accuracies of 95.80 and 91.98% respectively.

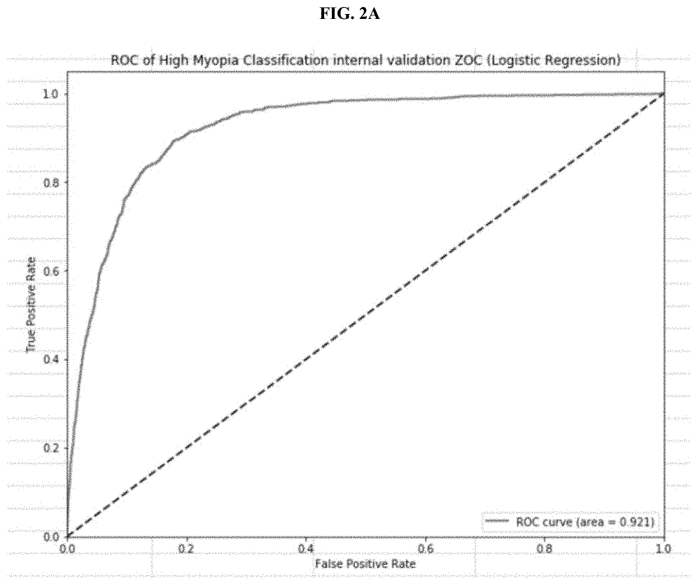

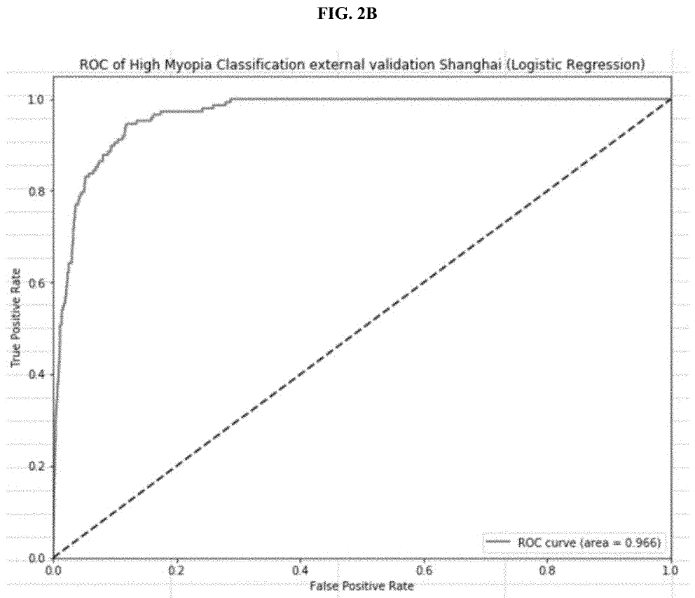

ROC curves for the three cohorts can be seen in FIG. 2. In the Guangzhou Myopia Study dataset, the area under the curve (AUC) was calculated to be 0.921, meaning the logistic regression model for predicting progression to high myopia in this dataset had a probability of 92.1%. For Shanghai Children Eye Study, AUC was calculated to be 0.966.

Myopia Control Study

The mean age at the time of baseline was 10.54 years of age (STD: 2.75 years). The mean SE at baseline was −3.61 (STD: 1.89). The mean time of follow-up was 3.62 (STD: 1.48).

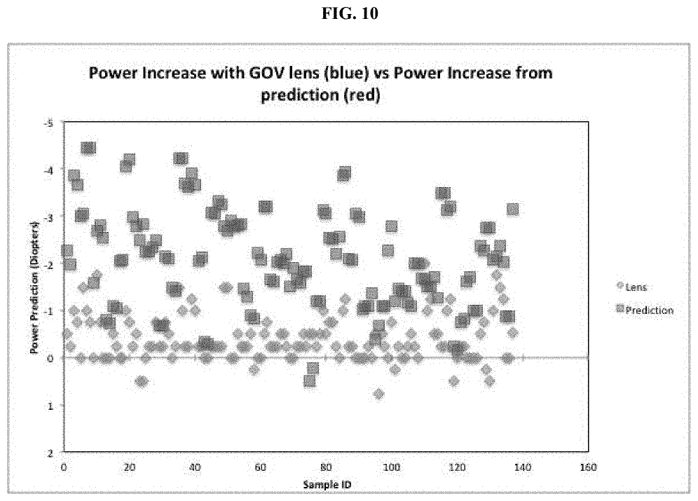

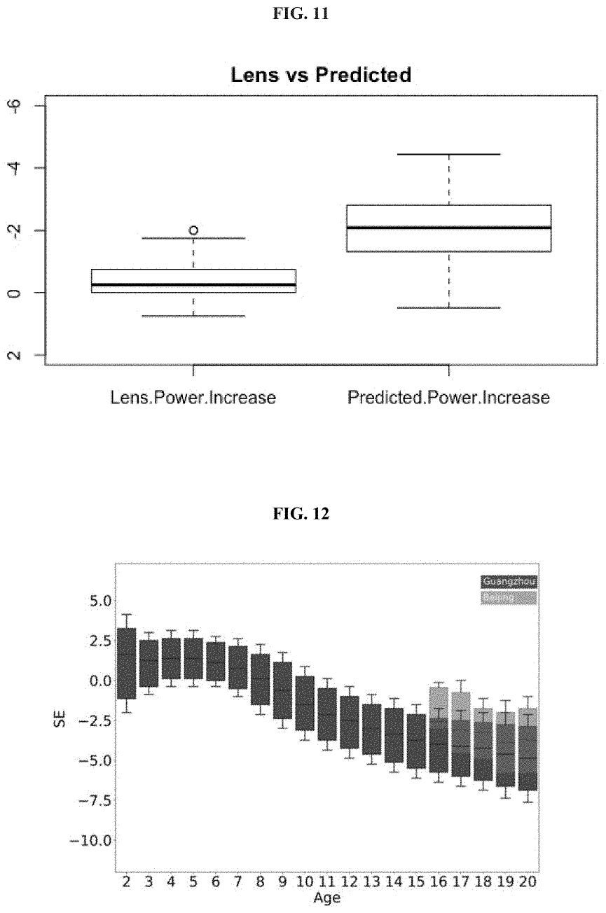

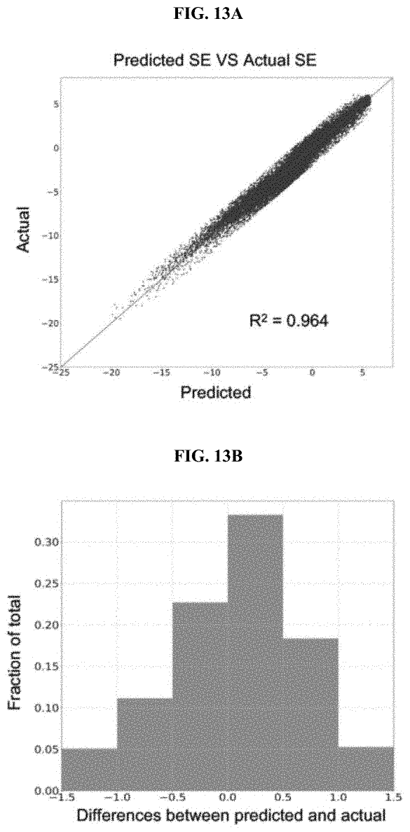

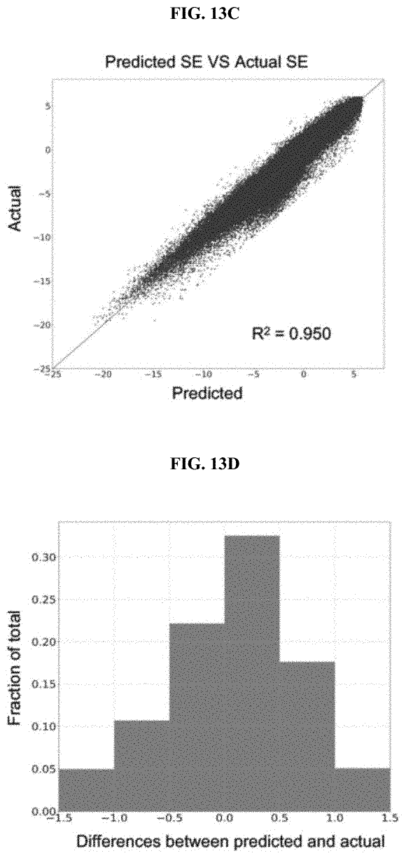

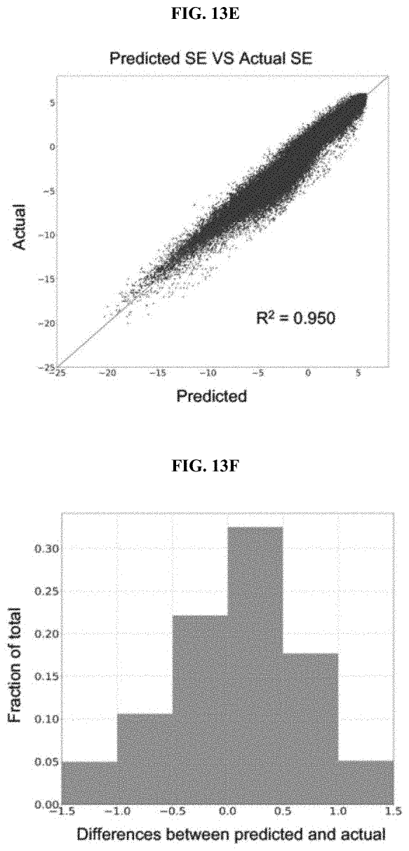

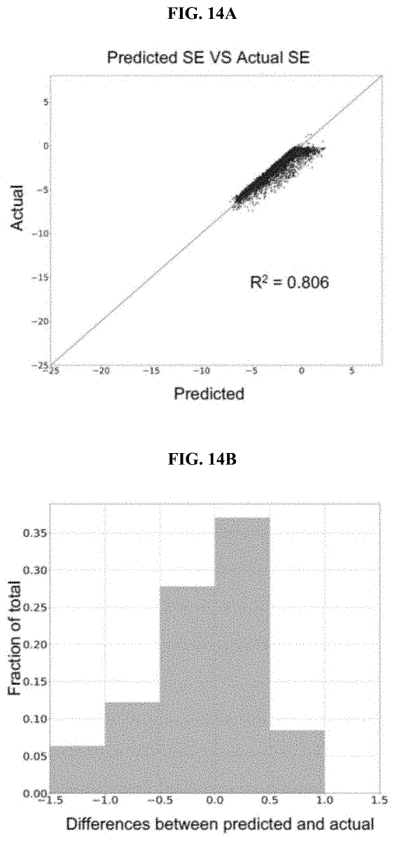

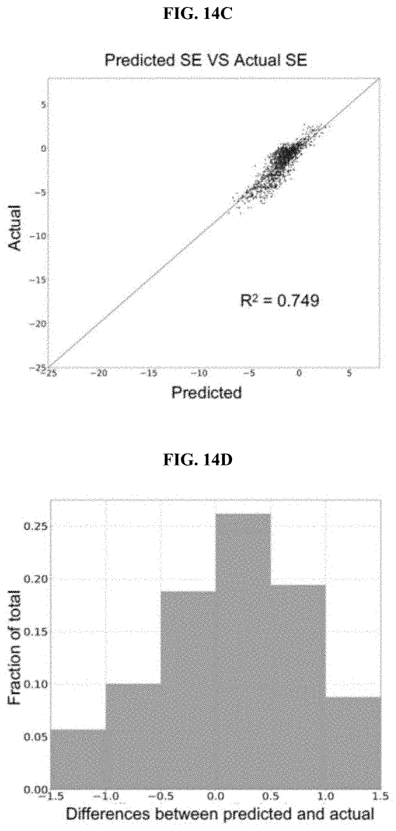

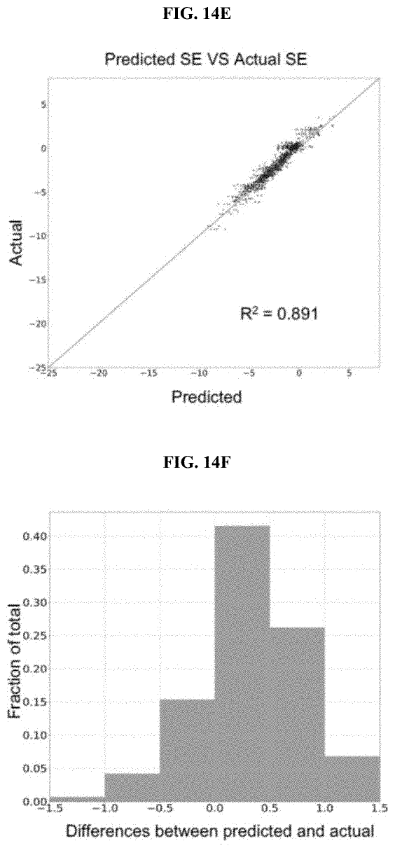

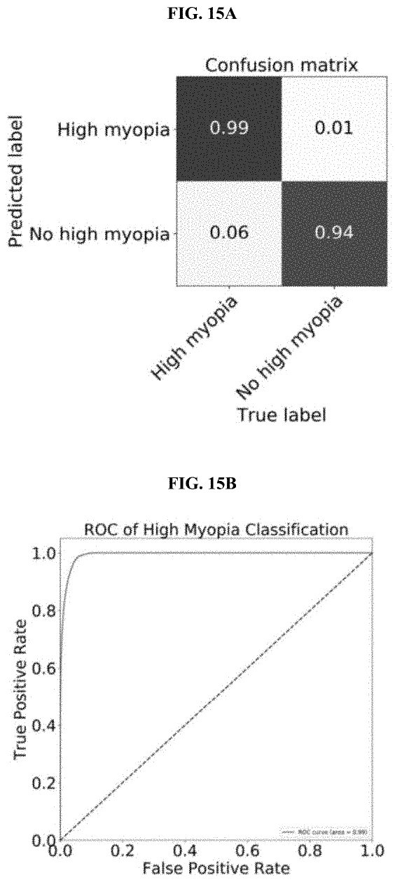

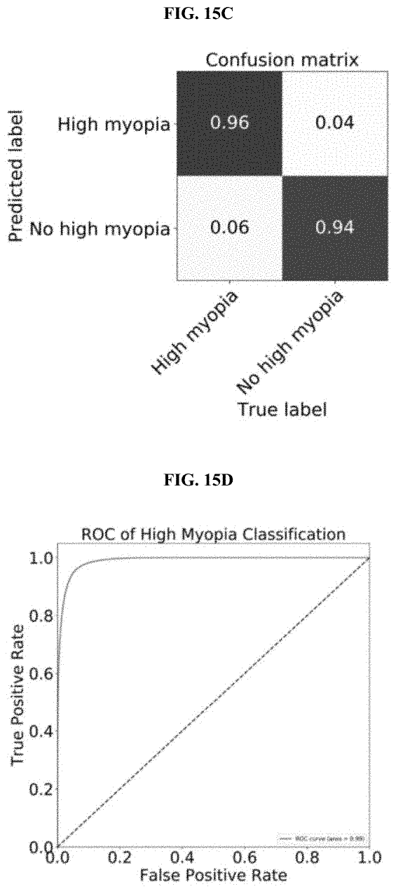

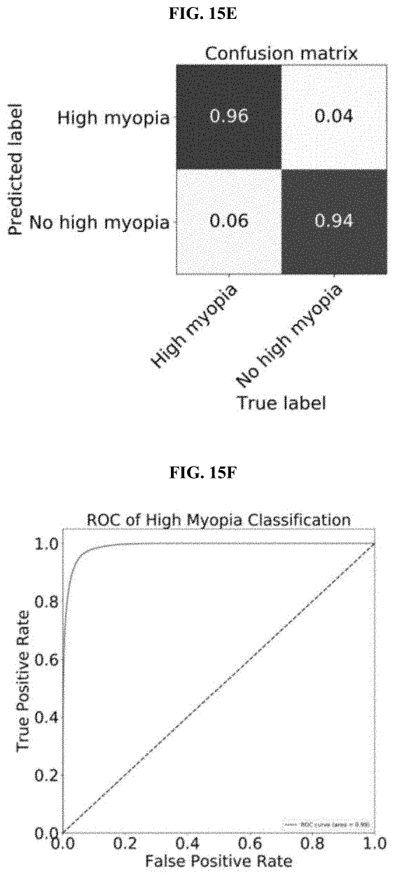

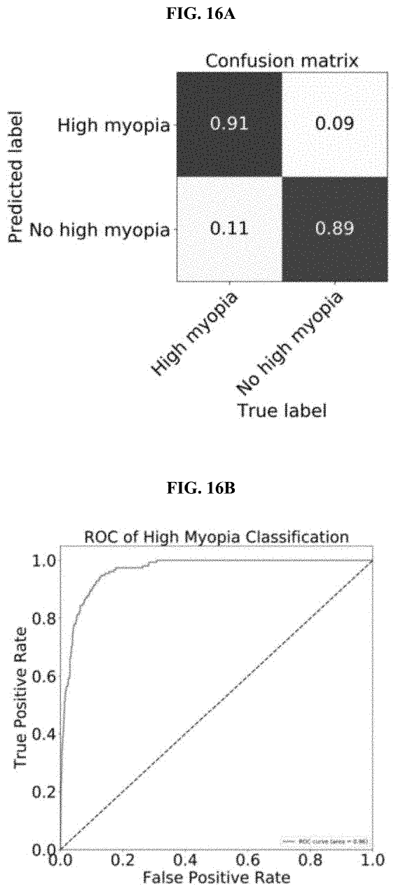

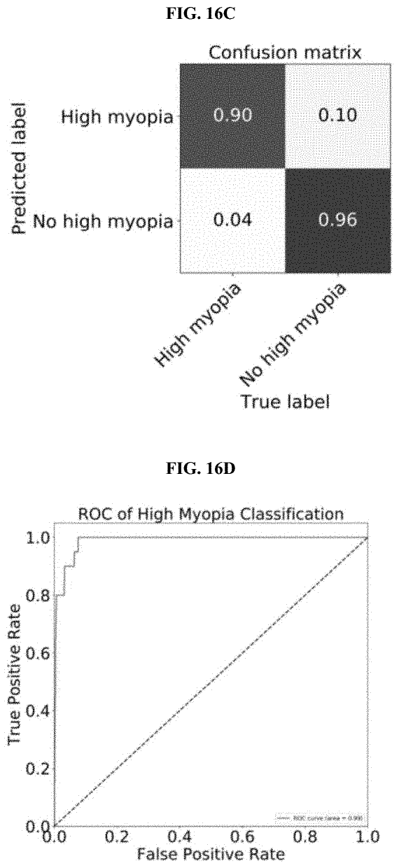

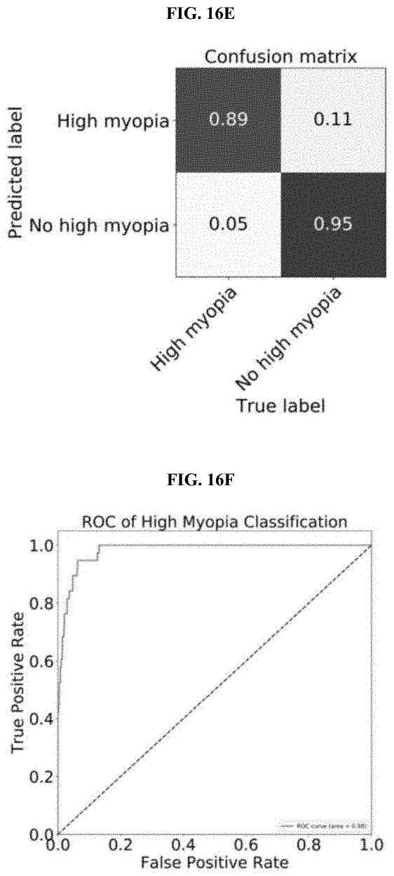

The actual power increase after wearing the lenses was compared to the predicted power increase using a Welch's two-sample independent t-test. The results were a t-value of 17.009, degrees of freedom of 196.73, and the p-value was less than 2.2e-16. The mean of the power increase after wearing lenses was −0.41 (STD: 0.51), while the mean predicted power increase was −2.11 (STD: 1.04) (FIG. 10 and FIG. 11).

Atropine Use Study

Genetic and environmental factors are thought to contribute to the development of myopia. Myopia can be either syndromic or non-syndromic and is generally a polygenic disease. In non-syndromic cases, genetic mapping has shown at least 16 independent loci (MYP2-MYP17) carrying different degrees of myopic severity with a number of candidate genes associated to the disease, such as those related to the maintenance of the extracellular matrix (e.g., collagen, actin, etc.), which leaves nearly 70 genes directly or indirectly linked to myopia.

Although genetic predispositions play a vital role in the pathogenesis of myopia, new sociocultural and environmental factors have shown some correlation. Most notably is the difference in prevalence between rural and urban areas. In two separate studies conducted by Sapkota and Pokherel, myopia prevalence of 15-year old Nepalese children living in urban districts was 27.3% comparative to less than 3% for those living in rural areas. Similar trends have been reported across Chinese and Indian populations.

Within the past several decades the advent of the internet and other associated technological advancements have simultaneously led to a “visual near work” era in which the influx of computer use and online platforms have subsequently caused accommodation spasms.

### Example 2

Data Collection

Collection of 613,839 medical records of 227,928 patients from five Chinese cohorts and one U.S cohort was carried out (Table 2). All cohorts were composed by infants to young adults that underwent complete eye exam at various time points. 273,307 clinical records of 88,111 patients (49,993 male; 38,118 female) with two or more visits from the Guangzhou Myopia Study (GMS) cohort were used to train the AI system. 29,445 medical records of 10,023 patients (5,778 male; 4,245 female) were used as the internal validation set. Clinical demographics for each cohort are listed in Table 2. All other medical records from the remaining five cohorts were used to externally validate our ML model described in this study.

The data was collected from the following six independent cohorts: Guangzhou Myopia Study (GMS) from Zhongshan Ophthalmic Center of Sun Yat-Sen University and Guangzhou Women and Children's Medical Center (302,752 medical records of 98,134 patients split 9:1 for training and internal validation; external validation 1: 203,462 medical records of 76,314 patients; external validation 2: 100,213 medical records of 50,957 patients), Shanghai Children Myopia Study (SCMS) from Eye and ENT Hospital of Fudan University (4,148 medical records of 2,007 pediatric patients), Beijing Children Eye Study from Beijing Tongren Eye Center (BCES) (1,955 medical records of 131 pediatric patients), and San Diego Children's Study from X (1,309 medical records of 385 patients). The characteristics of cohorts were summarized in Table 2.

The GMS data used for training provided a plethora of information, consisting mainly of bilateral eye exams in both children and young adults. The mean age at the time of baseline was 8.18 years old (standard deviation (SD): 3.54 years). The mean spherical equivalent (SE) refraction at baseline was −0.67 D (SD: 3.54 D). The mean interval between visits was 1.99 years (SD: 1.32 years). For internal validation set, the mean age at baseline, SE at baseline, and visit interval was 8.14 (SD: 3.52), −0,63 D (SD: 3.44 D), and 1.93 (SD: 1.26), respectively.

The first external validation consisted mainly of bilateral eye exams in both children and young adults from (study 1), with the mean age at the time of baseline to be 8.16 years old (SD: 3.54 years). The mean SE refraction at baseline was −0.67 D (SD: 3.45 D), and the mean interval between visits was 1.99 years (SD: 1.33 years). The second external validation also consisted of mainly of bilateral eye exams in both children and young adults from (study 2), with the mean age at the time of baseline to be 8.19 years old (SD: 3.56 years). The mean SE refraction at baseline was −0.66 D (SD: 3.45 D) and the mean interval between visits was 1.99 years (SD: 1.33 years)

The SCMS data consisted of bilateral eye exams in children with mainly one follow-up visit typically within a year after baseline. In this cohort, the mean age at baseline was 7.91 years (SD: 3.19 years). The mean SE refraction at baseline was −2.03 D (SD: 1.81 D). The mean interval between visits was 0.72 years (SD: 0.21 years).

The BCES cohort was composed of primary school-aged children with six annual follow-ups between 2011 and 2016; refraction was measured only for the right eye at each visit. In this cohort, the mean age at baseline was 7.71 years (SD: 1.36 years). The mean spherical equivalent refraction of the right eyes at baseline was −0.50 D (SD: 1.37 D). The mean time period between visits was 2.47 (SD: 1.26).

Lastly, the SDCS cohort was composed of X between A-year and B-year; ocular refraction was measured at each visit. In this cohort, the mean age at baseline was 10.65 years (SD: 2.34 years). The mean spherical equivalent refraction of the eyes at baseline was −1.58 D (SD: 1.91 D). The mean time period between visits was 1.55 (SD: 0.97).

The spherical equivalent was calculated from refractive error, defined by the following equation:

Spherical Equivalent=Spherical Diopter+½ (Cylindrical Diopter)

The annual progression of refractive error was calculated for each visit using the equation below:

Progression=(Spherical Equivalent]_{baseline}−Spherical Equivalent)/(t_{baseline}−t)

### Example 4

Machine Learning Training

Machine Learning Algorithms were Trained on the GMS Database

For training purposes, raw data were curated to include only patients meeting the following criteria: two or more visits; younger than 20 years old at the baseline visit; follow-up periods at least six months apart; baseline SE between 6.00 D and −20.00 D with presence of myopic progression, defined as endpoint SE less than baseline SE. Myopia progression greater than 3 diopters per year were considered atypical and excluded. The same inclusion criteria were applied to all five external validation data sets.

Applications of Regression and Classification Algorithms, Training and Validation

Two distinct learning methods, regression and classification, were used to develop the ML models. A Multivariate linear regression was used to analyze the linear relationship between two or more explanatory variables. This model was applied to the data from Example 2 to predict annual myopia progression by exploring the linear relationship between the following features: age at baseline, SE at baseline, the time interval between baseline and follow-ups, and corresponding outputs: SE at subsequent follow-up sessions.

The same features were applied to logistic regression, a form of classification, to determine if a patient would progress to high myopia, defined as SE<−6.00 D. In implementing this form of ML, the study not only accurately predicted progression, but also flagged patients susceptible to later onset of high myopia.

GMS data were used for training and internal validation. Patients were split into a 9:1 training-to-testing ratio and simultaneously evaluated using 10-fold cross-validation. External validation was performed in five other independent cohorts to confirm the rate of progression.

The metrics used to evaluate the multivariate linear regression model with internal validation for the GMS cohort and external validation for the GMS1, GMS2, SCES, BCMS and SDCS cohorts were mean absolute error (MAE) and R2 value. MAE is calculated by taking of absolute errors, and is better applied when needing to distribute equal weight across all errors. To evaluate the efficacy of logistic regression as a classification model of predicting high myopia cases, the following metrics were used: accuracy, sensitivity of predicting high myopia, specificity of identifying non-progressors, and area under the ROC curve (ROC-AUC).

### Example 3

Determining Myopia Distribution

When assessing the myopic distribution of the training cohort from Example 2, a noticeable correlation was observed between age and spherical equivalent (SE) from age 2 year old to age 20 year old (FIG. 12). GMS younger children population showed mainly hyperopic to plano refraction errors. At age 8 years old, the median SE observed was 0,125 D (25th percentile [Q1] being −1,50 D and 75th percentile [Q3] being 1,625 D); at age 16 years old, the median SE was −4,00 D (Q1=−5,75 D and Q3=−2,375 D); finally, at age 20 years old, the median SE observed was −4,875 D (Q1=−6,875 D and Q3=−2,875 D). These results suggest the presence of myopia progression over time. Similarly, when analyzing SE data from undergraduate students from Beijing, a median SE of −2,56 D (Q1=−2,96 D and Q3=−0,4375 D) was observed in patients at age 16 years old and median SE of −3,75 D (Q1=−5,75 D and Q3=−1,75 D) at age 20 years old (FIG. 12). Furthermore, this progression trajectory is similar to other demographic population studies reported in the literatures, illustrating the applicability of the model (2).

### Example 4

Myopia Progression Prediction

Multivariate linear regression was used to analyze progression of SE over time for the data from Example 2. Scatter plot graphs of actual and predicted SE for each instance in all cohorts and histograms of prediction error were generated to further evaluate the accuracy of the model for each cohort.

The model produced high accuracies across all cohorts while fitting the variability of each dataset. The internal validation of the GMS dataset produced an R-squared (R2) value of 0.964 (FIG. 2A), and mean absolute error (MAE) of 0.119 [95% CI: 0.119, 1.146], with predicted values within 1 D from actual SE 86% of the time (FIG. 2B).

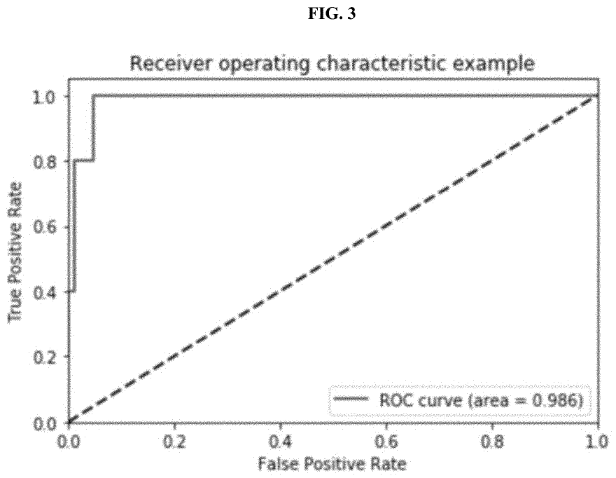

Next, the model was tested on external cohorts from China. When tested in study 1 dataset, the model produced an R2 value of 0.950 (FIG. 2C) and a MAE of 0.119 D [95% CI: 0.119, 1.136], with predicted values within 1 D 83% of the time (FIG. 2D); whereas in study 2, the model produced an R2 value of 0.950 (FIG. 2E) and a MAE of 0.121 D [0.121, 1.144], with predicted values within 1 D 83% of the time (FIG. 2F). A third external validation using the Shanghai Children Eye Study (SCES) cohort was also performed, which produced an R2 value of 0.806 (FIG. 3A) and a MAE of 0.066 D [−0.066, 0.569], with predicted values within 1 D 86% of the time (FIG. 3B). Furthermore, a fourth external validation using the Beijing Children Eye Study (BCES) produced an R2 value of 0.749. (FIG. 3C) with a MAE of 0.178 D [0.178, 1.557] and predicted values within 1 D 74% of the time (FIG. 3D). Lastly, the myopia progression model was evaluated in a U.S children cohort to determine the general applicability of our model in another geographically distinct population. The model produced an R2 value of 0.891 (FIG. 3E) with a MAE of 0.358 D [0.358, 1.456], with predicted values within 1 D 87% of the time (FIG. 3F). Therefore, despite differences in prevalence rates between Chinese and U.S populations, the model was able to accurately predict SE over time in both different geographic populations.

### Example 5

Predicting High Myopia

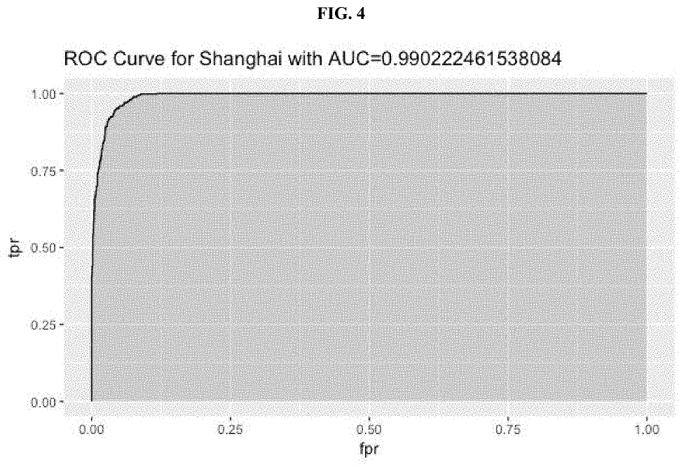

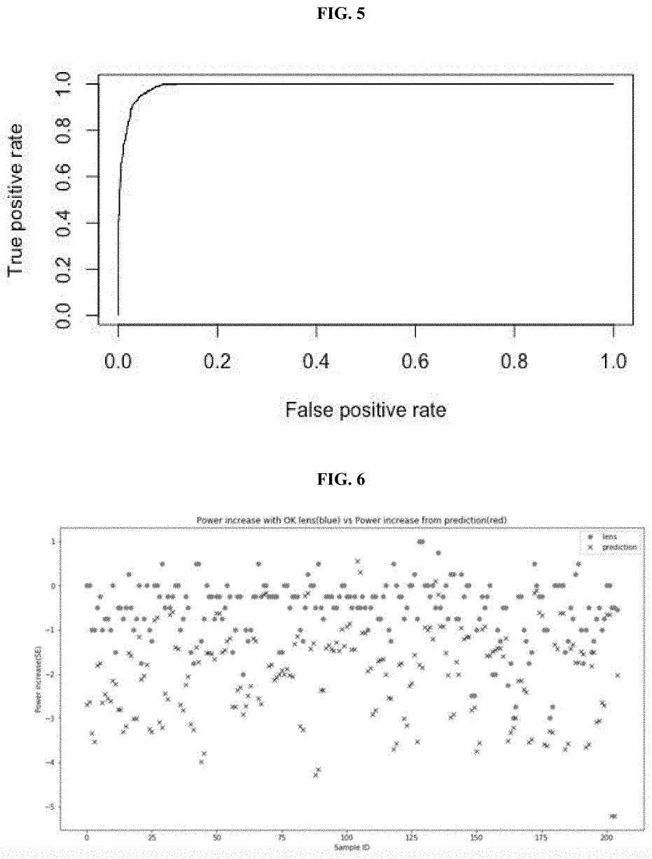

High myopia has been associated with a range of comorbidities, such as glaucoma and maculopathy, further elevating the severity of the disease. In addition, it places significant financial and healthcare strains on working population, given the direct and indirect costs associated with vision loss. To predict the onset of high myopia, an AI algorithm was trained to predict which patients would progress to high myopia (defined by SE<−6.00 D) using the data from Example 2. Logistic regression classifiers were trained to detect cases that would progress to high myopia and Receiver Operating Characteristic (ROC) curves were built for all six cohorts. In the internal validation cohort, the model produced an accuracy of 94.31%, sensitivity of 98.96%, and specificity of 93.70% (FIG. 4A) in detecting high myopia progressors, and area under the curve (AUC) of 0.99, indicating high accuracy (FIG. 4B). External validation also yielded similar results. When evaluating the study 1 dataset, the model produced an accuracy of 93.95%, sensitivity of 96.28%, specificity of 93.62% (FIG. 4C) and AUC of 0.99 (FIG. 4 D). Similar results were observed when classifying high myopia in study 2 dataset: logistic regression produced an accuracy of 93.93%, with sensitivity of 96.29%, specificity of 93.59% (FIG. 4E) and AUC of 0.99 (FIG. 4F). When evaluating the SCES cohort, the model achieved an accuracy of 89.54%, sensitivity of 90.91%, specificity of 89.49% (FIG. 5A) and AUC of 0.96 (FIG. 5B). Similarly, an accuracy of 96.18%, sensitivity of 90.00%, specificity of 96.27% (FIG. 5C) and AUC of 0.99 (FIG. 5 D) were produced when evaluating the BCES dataset. Lastly, the classifiers were validated in the SDCS dataset, producing an accuracy of 89.54%, sensitivity of 90.91%, specificity of 89.49% (FIG. 5E) and AUC of 0.98 (FIG. 5F).

1. A computer-implemented method for predicting myopia progression in an individual, comprising:
a) obtaining data of the individual;
b) evaluating the data using a machine learning algorithm to generate a prediction of myopia progression; and
c) providing the prediction to the individual or a third party.

2. The method of claim 1, wherein the machine learning algorithm comprises a linear model providing a relationship between myopia progression and two or more features.

3. The method of claim 1, wherein the machine learning algorithm is generated using a machine learning procedure comprising multivariate linear regression analysis.

4. The method of claim 1, wherein the machine learning algorithm comprises a logistic regression model.

5. The method of claim 1, wherein the machine learning algorithm comprises a support vector machine model.

6. The method of claim 1, wherein the prediction comprises a likelihood of progression to a higher degree of myopia.

7. The method of claim 6, wherein the higher degree of myopia is low myopia, moderate myopia, or high myopia.

8. The method of claim 6, wherein the low myopia corresponds to a spherical equivalent of −3.00 diopters or less.

9. The method of claim 6, wherein the moderate myopia corresponds to a spherical equivalent between −3.00 diopters to −6.00 diopters.

10. The method of claim 6, wherein the high myopia corresponds to a spherical equivalent of −6.00 diopters or more.

11. The method of claim 1, wherein the individual has myopia.

12. The method of claim 1, wherein the individual does not have myopia.

13. The method of claim 1, wherein the prediction comprises an age of onset of myopia in an individual who does not have myopia.

14. The method of claim 1, wherein the prediction comprises a rate of progression of myopia.

15. The method of claim 14, wherein the rate of progression comprises a predicted change in degree of myopia within a time period.

16. (canceled)

17. (canceled)

18. The method of claim 1, wherein the prediction comprises a predicted change in myopia for one or both eyes of the individual.

19. The method of claim 1, wherein the data comprises an age and refraction under cycloplegia of one or both eyes of the individual measured at one or more time points.

20. (canceled)

21. (canceled)

22. The method of claim 1, wherein the prediction has an accuracy of at least 80% when tested against an independent data set of at least 200 samples.

23. The method of claim 1, further comprising providing a recommendation for treatment or providing a treatment to the individual or the third party, wherein the treatment comprises atropine eye drops or orthokeratology.

24.-25. (canceled)

28. The method of claim 1, further comprising performing further testing on the individual, wherein the further testing comprises an optometry exam, genetic testing, imaging one or both eyes of the individual, screening for ocular disorders or conditions, or any combination thereof.

29.-104. (canceled)
